SEQUENCE LISTING (7 sequences)

SEQ ID NO: 1 - protein, 723 aa, unidentified
  source 1..723: note = AAV5 VP1
SFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 2 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTDKPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 3 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTGKPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 4 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLFRFVSTDATGNLKFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 5 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLSAGGNRNYLSAKATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 15 aa, synthetic construct
LFRFVSTDATGNLKF

SEQ ID NO: 7 - protein, 14 aa, synthetic construct
LSAGGNRNYLSAKA

(19) United States
(12) Patent Application Publication
(10) Pub. No.: US 2025/0084129 A1
(43) Pub. Date: Mar. 13, 2025

(54) AAV5 CAPSID VARIANTS

(71) Applicant: University of Florida Research Foundation, Incorporated, Gainesville, FL (US)

(72) Inventors: Oleksandr Kondratov, Gainesville, FL (US); Anatolii Mamchur, Gainesville, FL (US); Maria Ukhanova, Gainesville, FL (US); Sergei Zolotukhin, Gainesville, FL (US)

(73) Assignee: University of Florida Research Foundation, Incorporated, Gainesville, FL (US)

(21) Appl. No.: 18/727,527

(22) PCT Filed: Jan. 10, 2023
(86) PCT No.: PCT/US2023/060403

§ 371 Date: Jul. 9, 2024

Related U.S. Application Data
(60) Provisional application No. 63/298,139, filed on Jan. 10, 2022.

(51) Int. Cl. C07K 14/005; C12N 15/86

(52) U.S. Cl. CPC C07K 14/005; C12N 15/86; C12N 2750/14122; C12N 2750/14143

(57) ABSTRACT

Disclosed herein are recombinant AAV variant (e.g., variant serotype 5 (AAV5)) capsid proteins and variant capsid protein-containing viral particles with enhanced ability to transduce adult stem cells and neurons. Viral particles containing these capsid variants are capable of enhanced transduction of mammalian mesenchymal stem cells and neurons. The recombinant AAV5 variant proteins and viral particles disclosed herein were identified from a variant AAV5 capsid library that was engineered by making substitutions in variable regions of the capsid. Compositions of these variant AAV5 particles are provided that are useful for transducing and delivering therapeutic transgenes to cells, such as mesenchymal stem cells, and thus treat diseases and disorders pertaining to these cells.

### RELATED APPLICATIONS

This application claims priority under 35 U.S.C. § 119(e) to U.S. Provisional Application, U.S. Ser. No. 63/298,139, filed on Jan. 10, 2022, which is incorporated herein by reference.

### GOVERNMENT SUPPORT CLAUSE

This invention was made with government support under Grant Number R01 HL097088, awarded by the National Institutes of Health. The government has certain rights in the invention.

### REFERENCE TO AN ELECTRONIC SEQUENCE LISTING

The contents of the electronic sequence listing (U119670094WO00-SEQ-PRW.xml; Size: 10,915 bytes; and Date of Creation: Jan. 9, 2023) is herein incorporated by reference in its entirety.

### BACKGROUND

Recombinant adeno associated virus (rAAV) vectors are among the most promising tools for in vivo human gene therapy, as demonstrated by an increasing number of clinical trials as well as treatment approvals being reported worldwide¹. Their favorable safety profile (especially at lower vector doses) due to the episomal localization of the therapeutic gene (non-integrating vector), minimal issues of insertional mutagenesis², and lack of pathogenicity in transduced cells make these small virions the gene delivery vectors of choice. The ability of rAAV vectors to transduce long lived non-dividing cells such as myocytes or hepatocytes also makes them ideal for sustained expression of transgenes, which would otherwise get diluted upon cell division. Clinical translation of rAAV therapy has so far been successfully applied for hereditary blindness (Luxturna®), neuromuscular disorders (Zolgensma®), and coagulation disorders (hemophilia B), among others³⁻⁵.

Adeno-associated virus (AAV) is a single-stranded DNA virus belonging to the Parvoviridae family (Muzyczka & Berns, 2001). AAV-derived vectors are promising tools for human gene therapy applications because of their absence of pathogenicity, low immunogenicity, episomal localization and stable transgene expression. However, significant limitations to the clinical use of AAV are its promiscuity and its susceptibility to neutralization by human antibodies (Jeune et al., 2013). Both of these limitations are determined by nature of the amino acid residues exposed at the surface of the capsid. Therefore, major efforts aiming at developing useful and effective gene therapy vectors have been devoted to obtaining and studying capsid variants (Wu et al., 2006). The first approach was to study naturally occurring AAV isolates. Thus far, 13 serotypes have been formally characterized and hundreds of variant isolates have been sequenced. Additional capsid variation has been investigated through the generation of mosaics (viral particles made of capsid proteins from more than one serotype) (Hauck et al., 2003; Stachler and Bartlett, 2006; Gigout et al., 2005), chimeras (capsid proteins with domains from various origins) (Shen et al., 2007), and various substitutional or insertional mutants (Wu et al., 2000). However, the most significant advances are expected to result from directed evolution approaches through the development of capsid libraries.

The state of the art method for randomizing an AAV capsid-encoding genetic sequence was until recently error-prone polymerase chain reaction (PCR), which introduced randomly dispersed mutations throughout the roughly 730 amino acids that constitute the AAV capsid sequence. However, the error-prone PCR technique suffered from two key problems. First, random mutagenesis often installed mutations that were deleterious to capsid function, as only 0.01-1% of mutations were typically beneficial (see, e.g., Romero & Arnold (2009), Nat Rev Mol Cell Biol 10(12): 866-876 and Guo, Choe & Loeb (2004), Proc Natl Acad Sci USA 101(25): 9205-9210.). Second, the sheer number of PCR clones that needed to be generated to cover all combinations of multiple mutations within a single capsid by far exceeded the technical capabilities of the skilled artisan. For instance, it has been calculated that, to comprehensively randomize five residues within a 414 base pair fragment of the AAV2 capsid VP1 gene, an AAV library would have to comprise nearly 10¹¹ different clones to cover a single mutation at each site (see Maersch et al. (2010), Virology 397(1): 167-175).

The various strategies to generate capsid libraries that have been developed so far all suffer from sequence bias or limited diversity. Random display peptide libraries (Govindasamy et al., 2006) are limited to an insertion at one particular capsid location. Libraries generated using error-prone polymerase chain reaction (PCR) contain a very small fraction of gene variants encoding proteins that can fold properly and assemble into a functional capsid, due to the randomness of the mutations. DNA shuffling and staggered extension processes are more efficient because they recombine naturally-occurring parental sequences and therefore are more likely to generate actual capsid variants. However, they can only recombine blocks of DNA as opposed to single nucleotide positions, which results in sequence bias (parental polymorphisms will tend to cluster together instead of being randomly distributed).

### SUMMARY OF THE INVENTION

The present disclosure provides adeno-associated virus (AAV) capsid variants and virions comprising capsid variants that exhibit enhanced transduction of target mammalian cells, such as mesenchymal stem cells. In some aspects, the present disclosure provides modified capsids of serotype 5, also known as modified AAV5 capsids or AAV5 capsid variants.

In some aspects, the present disclosure provides the AAV5-DK capsid protein variant, or “DK.” In some aspects, the present disclosure provides the AAV5-GK capsid variant, or “GK.” Each of DK and GK contains two amino acid substitutions compared with wild-type AAV5. These substitutions are at residues 377 and 378.

In some aspects, the present disclosure provides the AAV5-FDA capsid variant, or “FDA.” In some aspects, the present disclosure provides the AAV5-SAG capsid variant, or “SAG.” The FDA variant contains six amino acid substitutions compared with wild-type AAV5. The SAG variant contains seven amino acid substitutions compared with wild-type AAV5.

The present disclosure is based, at least in part, on the rational generation of AAV capsid variant libraries through the introduction of motifs of novel mutations in the native capsid through mutagenesis and directed evolution. The present disclosure is further based on the screening of variants from amongst one such library of AAV5 capsids. According to the present disclosure, molecular evolution using a combinatorial library platform has generated capsid variants with high tropism for mesenchymal stem cells (MSCs). These capsid variants have also demonstrated high tropism for mammalian neurons.

MSCs are adult stem cells which can be isolated from human and non-human animal sources. Human MSCs (hMSCs) are non-haematopoietic, multipotent stem cells with the capacity to differentiate into mesodermal lineage cells, such as osteocytes, adipocytes and chondrocytes, as well ectodermal (neurocytes) and endodermal lineages (hepatocytes). MSCs express cell surface markers like cluster of differentiation (CD)29, CD44, CD73, CD90, CD105 and lack the expression of CD14, CD34, CD45 and HLA (human leukocyte antigen)-DR. hMSCs for the first time were reported in the bone marrow and have since also been isolated from adipose tissue, amniotic fluid, endometrium, and dental tissues, among other tissues. Bone marrow-derived MSCs (BM-MSCs) are considered the best source for MSCs. MSC cultures are self-renewable, multipotent, and, crucially, expandable in vitro with exceptional genomic stability.

Tissue-specific MSCs are known to be widely distributed in perivascular regions of almost all tissues throughout the body, where they are thought to play an important role in tissue homeostasis, physiological remodeling, injury repair, and tissue regeneration throughout the life of the subject. See Almeida-Porada, Atala & Porada, Mol. Ther Meth. & Clin. Dev. 16:204-224 (2020), which is incorporated herein by reference. MSCs have the capacity to migrate and seed specifically into damaged tissue sites, where they can replace damaged or diseased cells via differentiation and/or reprogramming in situ and to secrete cytokines, proteolytic enzymes, and/or angiogenic and trophic factors that stimulate the proliferation and survival of endogenous cells within the local tissue.

MSCs have immunomodulatory properties that facilitate their use in cell therapy, ex vivo treatments, and transplantation. Due to low expression of MHC I, and a lack of expression of MHC class II and co-stimulatory molecules CD80, CD40 and CD86, MSCs do not elicit substantial alloreactivity. Lack of expression of these molecules protects MSCs from attack by cytotoxic T cells. Of particular clinical interest, MSCs also exhibit a remarkably potent ability to skew the balance between effector/memory T cells and CD4+ FoxP3+ regulatory T cells (Tregs), polarizing both naive and memory T cells toward a Treg phenotype in vitro and mediating the immune response toward tolerance. Due to their immunomodulatory properties, MSCs have been used in many autoimmune disease clinical trials. In particular, preclinical and clinical studies have been performed using hMSCs in treatment of chronic diseases like neurodegenerative, autoimmune and cardiovascular diseases. MSCs have the ability to differentiate into neurons, and as such have been used in transplantations for neurodegenerative disorders. For example, MSCs have been evaluated as immunotherapies to treat autoimmune disorders, inflammatory bowel disease (IBD), type 1 and type 2 diabetes, arthritis, ischemia-reperfusion injury; and therapies to thwart immunological complications that arise following the transplantation of stem cells, organs, and allografts.

Importantly, MSCs are ideal vehicles for gene delivery, as they can be transduced at high efficiency with major viral-based vectors, including AAV. Following transduction, the gene-modified MSCs can be selected and extensively expanded in vitro to generate adequate numbers for transplantation. This is in marked contrast to HSCs, which cannot be expanded in vitro without loss of in vivo functionality. The immunomodulatory nature of MSCs also represents a significant advantage of their use in gene therapy, as it may enable MSCs expressing a “foreign” cargo to go undetected by the recipient's immune system. The use of allogeneic gene-modified MSCs should thus be possible. MSCs have been used as cellular vehicles for delivering Factor XIII and Factor IX to subject suffering from hemophilia A. In addition, human MSCs engineered to express and secrete interferons have been evaluated in suppressing tumor progression, such as brain tumors.

The development of next-generation rAAV viral particles, or virions, may dramatically reduce the number of viral particles needed for a conventional gene therapy regimen. In addition to having improved transduction efficiencies for various mammalian cells, the rAAV virions prepared as described herein may be more stable, less immunogenic, and/or can be produced at much lower cost, or in a higher titer, than an equivalent wild type viral vector prepared in conventional fashion.

In the practice of the present disclosure, native amino acids normally present in the sequence of a viral capsid protein, such as a wild-type capsid of serotype 5, may be substituted by one or more non-native amino acids, including substitutions of one or more amino acids not normally present at a particular residue in the corresponding wild-type protein. In some embodiments, the amino acid substitutions in the disclosed capsid variants may be epistatic (interacting) with respect to one another. These amino acid substitutions may act synergistically on capsid binding and transduction. In some embodiments, the amino acid substitutions comprise one or more motifs.

In some embodiments, the amino acid substitutions in the disclosed capsid variants confer upon virions comprising these variants an enhanced ability to evade neutralizing antibodies of the host immune system. In some embodiments, the disclosed virions have reduced seroreactivity. In some embodiments, the disclosed virions are able to evade the humoral immune response, e.g., neutralizing antibodies, of a subject following their delivery into the subject. In particular embodiments, the subject is mammalian. The subject may be human. The subject may be a non-human primate.

Wild-type AAV is a small (˜26 nm), non-enveloped parvovirus. It packages a linear single-stranded DNA genome (˜4.7 kb), encoding genes necessary for replication (rep) and the viral capsid (cap), flanked by palindromic inverted terminal repeats (ITRs). Except for the ITRs which are essential, much of the viral DNA genome can be omitted for the purpose of transgene packaging and delivery, allowing for insertion of approximately 4.7 kb of foreign DNA, which altogether forms the transgene expression cassette⁶. In some cases, a self-complementary single strand duplex DNA can be packaged⁷, although this reduces the transgene capacity to less than half, and increases the risk of immune response⁸.

There are several challenges that impede the successful and broad use of rAAV gene therapy. The first major limitation to systemic or intramuscular administration of AAV is the presence of pre-existing neutralizing antibodies (NAb) against the vector capsid that can block cellular entry⁹. A majority of the human population are seropositive for AAV, mostly due to previous subclinical exposure to the wild-type virus¹⁰. This is a major exclusion criterion for prospective patients, as even very low titers of NAb in circulation can prevent vector entry and significantly limit effective gene transfer to the target organ. Pre-existing anti-AAV NAb do not affect rAAV injection into immune privileged sites such as the eye or brain. Luxturna® (voretigene neparvovec-rzyl), the FDA approved drug for treating inherited retinal disorders, can be successfully delivered into the eye using AAV serotype 2, which is seroprevalent in 40-70% of the human population¹⁰⁻¹². The impact of low titer NAb (<1:5), particularly on systemic gene transfer is not accurately known¹³⁻¹⁵. Using a different serotype is complicated because a pattern of cross reactivity commonly occurs between variants, such as between AAV2, AAV5 and AAV8¹⁶, or AAV1 and AAV6¹⁷, depending on the degree of homology between capsid protein sequences.

A second limitation is transduction efficiency of target cells. Based on specific receptor interaction and post-entry mechanisms, AAV serotypes differ in cell tropism as well as transduction efficiency in the target cell type¹⁸. A major challenge to gene therapy is that the functional gene may not transduce the tissue in high enough numbers to provide therapeutic benefit. Increasing the rAAV dose in this case is not always effective, as a high viral load can induce detrimental capsid-specific T cell immune responses to the transduced cell^{14, 19}.

These limitations cannot adequately be addressed by the current repertoire of naturally occurring AAV serotypes, and the isolation and characterization of novel variants is time consuming. The nature of exposed amino acid residues on the capsid surface largely determines receptor attachment, tissue transduction, and antigenicity²⁰. Therefore, the development of engineered AAV vectors designed either by rational modification of specific amino acids (rational mutagenesis), or in vitro or in vivo selection in the cell type of choice (directed evolution) is an attractive alternative reported by Zolotukhin and others^{21,22}. These novel rAAV capsid vectors based on different serotypes have been shown to be superior at transducing the target cell type and in some cases, evading pre-existing NAb in the host²³⁻²⁶.

Unlike what would be expected from error-prone PCR, the novel mutations of the capsid variants of the present disclosure were not randomly or arbitrarily selected. In the present disclosure, rational mutagenesis and directed evolution strategies were combined to select for engineered AAV vectors derived from the AAV5 capsid backbone. In vitro, the presently disclosed engineered variants show improved tropism for human MSCs as compared to wild type (wt) AAV5. In vivo, the presently disclosed variants may show improved MSC transduction in human subjects. Further, the presently disclosed variants show improved neuron transduction in mammalian subjects in vivo. These variants may further exhibit reduced seroreactivity relative to, e.g., wild-type AAV5.

Certain embodiments of the modified AAV capsids and AAV virions of the present disclosure include the second nucleotide sequence encoding an AAV Cap protein that differs from wild-type serotype 5 VP1 capsid protein at least at one amino position. The at least one amino acid position that differs is preferably in a variable region (VR), and may be in variable regions 1, 4, 5, 6, 7, or 8 (VR-I, VR-IV, VR-V, VR-VI, VR-VII, VR-VIII) and combinations thereof. In some embodiments, the at least one amino acid position that differs is in variable region 5 or 6.

Certain aspects of the modified AAV capsids and AAV virions of the present disclosure comprise mutations in each of amino acid residues 377 and 378 in the wild-type AAV5 capsid sequence. These mutations may be made relative to the AAV5 VP1 capsid amino acid sequence set forth as SEQ ID NO: 1. In certain embodiments, the capsid protein comprises an N378K substitution. In some embodiments, the protein comprises an E377D substitution. In other embodiments, the protein comprises an E377G substitution. In some embodiments, the capsid comprises mutations in each of amino acid residues 377 and 378, and one, two, three, or more than three additional mutations.

In some embodiments, the protein comprises the amino acid sequence of SEQ ID NO: 2 (AAV5-DK). In some embodiments, the protein comprises the amino acid sequence of SEQ ID NO: 3 (AAV5-GK).

Certain aspects of the modified AAV capsids and AAV virions of the present disclosure comprise motifs of mutations in the AAV5 VP1 capsid protein. In some embodiments, the capsid comprises one of the following sequence motifs:
(a) LFRFVSTDATGNLKF (SEQ ID NO: 6) in variable region (VR) IV, or
(b) LSAGGNRNYLSAKA (SEQ ID NO: 7) in VR V.

In some embodiments, the variant rAAV5 capsid comprises mutations in at least one, at least two, at least three, at least four, at least five, or at least six of amino acid residues 436, 442, 443, 446, 447, and 448 in the wild-type AAV5 VP1 sequence of SEQ ID NO: 1. The capsid protein may comprise mutations at each of amino acid residues 436, 442, 443, 446, 447, and 448. In some embodiments, the protein comprises at least two, at least three, at least four, at least five, or at least six of the following substitutions: Y436F, N442D, N443A, G446N, V447L, and Q448K. The capsid protein may comprise each of the following substitutions: Y436F, N442D, N443A, G446N, V447L, and Q448K. In some embodiments, the capsid protein comprises the amino acid sequence of SEQ ID NO: 4 (AAV5-FDA).

In some embodiments, the variant rAAV5 capsid comprises mutations in at least one, at least two, at least three, at least four, at least five, at least six, or at least seven of amino acid residues 478, 479, 481, 484, 485, 486 and 489 in the wild-type AAV5 VP1 sequence of SEQ ID NO: 1. The capsid protein may comprise mutations at each of amino acid residues 478, 479, 481, 484, 485, 486 and 489. In some embodiments, the protein comprises at least two, at least three, at least four, at least five, or at least six of the following substitutions: G478S, S479A, V481G, A484N, S485Y, V486L, and F489K. The capsid protein may comprise each of the following substitutions: G478S, S479A, V481G, A484N, S485Y, V486L, and F489K. In some embodiments, the capsid protein comprises the amino acid sequence of SEQ ID NO: 5 (AAV5-SAG).

AAV virions containing any of the AAV5 capsid variants described herein are provided. Compositions comprising any of these AAV virions are provided. Pharmaceutical compositions comprising any of these AAV virions and a pharmaceutically acceptable carrier are further provided.

In some embodiments, the AAV virions of the present disclosure are incorporated into at least one host cell. Examples of suitable host cells are mammalian cells include MSCs, such as human MSCs and murine MSCs. Additional examples include human host cells, including, for example, blood cells, brain cells, myocardial cells, hematopoietic cells, CD34-expressing cells, liver cells, cancer cells, vascular cells, pancreatic cells, neural cells, glial cells ocular or retinal cells, epithelial or endothelial cells, dendritic cells, fibroblasts, or any other cell of mammalian origin, including, without limitation, adult stem cells (e.g., MSCs), hepatic cells, lung cells, cardiac cells, pancreatic cells, intestinal cells, diaphragmatic cells, renal (i.e., kidney) cells, bone marrow cells, or any one or more selected tissues of a mammal for which viral-based gene therapy is contemplated. In exemplary embodiments, the host cells are MSCs. In some embodiments, the host cells are neurons or glial cells.

AAV virions comprising the exemplary AAV5 variants of the present disclosure may include the virions as incorporated or transduced into at least one host cell. In particular embodiments, virions comprising the DK variant are incorporated into MSC cells. In particular embodiments, virions comprising the GK variant are incorporated into MSC cells.

In particular embodiments, virions comprising the FDA variant are incorporated into MSC cells. In particular embodiments, virions comprising the SAG variant are incorporated into MSC cells. In some embodiments, virions comprising the DK variant are incorporated into neurons or glial cells. In some embodiments, virions comprising the GK variant are incorporated into neurons or glial cells. In some embodiments, virions comprising the FDA variant are incorporated into neurons or glial cells. In some embodiments, virions comprising the SAG variant are incorporated into neurons or glial cells.

Embodiments of host cells comprising the AAV virions of the present disclosure further comprise a nucleotide sequence encoding at least one molecule providing helper function. The third nucleotide sequence may be a polynucleotide derived from an adenovirus or a herpes virus (e.g., HSV1). In particular embodiments, the polynucleotide is derived from adenovirus, e.g., Ad5.

In some aspects, the disclosure provides methods of selecting tissue-specific or cell-specific variants of an AAV virion includes (a) introducing a plurality of AAV virions into target tissues or cells; (b) allowing sufficient time to elapse to propagate additional virions; and (c) isolating the virions. Steps (a) through (c) may be repeated one or more times to enrich for a tissue-specific (e.g., MSC tissue-specific) or cell-specific variant. Such enriched variants exhibit a higher target tropism for the target tissues or cells, such as MSCs or neurons, as compared to AAV serotype 5.

Embodiments of the AAV virions of the present disclosure include (a) a first nucleotide sequence encoding at least one therapeutic molecule; (b) a second nucleotide sequence comprising a regulatory sequence; (c) a third nucleotide sequence comprising a first AAV terminal repeat (e.g., from serotype 5 or serotype 2); (d) a fourth nucleotide sequence comprising a second AAV terminal repeat (e.g., from serotype 5 or serotype 2); and (e) a capsid comprising at least one AAV Cap protein that differs from wildtype serotype 5 in at least at one amino acid position. In some embodiments, the AAV Cap protein that differs from wildtype serotype 5 in at least at two amino acid positions. The first nucleotide sequence is operably linked to the second nucleotide sequence and the first and second nucleotide sequences are interposed between the first and second AAV terminal repeat to form a transgene, and the resulting transgene is packaged within the capsid. Examples of suitable regulatory sequences include promoters and enhancers, e.g., an MSC tissue specific promoter. Examples of suitable therapeutic molecules include polypeptides, peptides, antibodies, antigen binding fragments, growth factors, cytokines and other small therapeutic proteins, and any combination thereof.

In some aspects, the present disclosure provides methods for treating a disease or disorder. Such methods may comprise administering an effective amount of an AAV virion of the present disclosure. In some embodiments, the disease or disorder is an autoimmune disorder, inflammatory bowel disease (IBD), type 1 diabetes, type 2 diabetes, arthritis, or ischemia-reperfusion injury. In some embodiments, the disease or disorder is an autoimmune disease. In some embodiments, the disease or disorder is a cancer or tumor of the brain.

The following drawings form part of the present specification and are included to demonstrate certain aspects of the present disclosure. The present disclosure may be better understood by reference to the following description taken in conjunction with the accompanying drawings, in which like reference numerals identify like elements.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows an alignment of the amino acid sequences of wild-type AAV5 against capsid variants DK, FDA, GK, and SAG (top to bottom). Substituted amino acids are bolded and underlined. The amino acid sequences that correspond to these capsid sequences are, from top to bottom, SEQ ID NO: 1, SEQ ID NO: 2, SEQ ID NO: 3, SEQ ID NO: 4, and SEQ ID NO: 5.

FIG. 2 shows a non-limiting example of vector genome (vg) quantification in an animal model for different AAV variants. Mice were injected with AAV variants as described in Example 2. Vector genomes were quantified in the forebrain, midbrain, and pons/medulla of each mouse. Vector genome levels are plotted by variant, from left to right variant A-variant H, for each brain region.

### DETAILED DESCRIPTION OF THE INVENTION

AAV-derived viral particles are promising tools for human gene therapy applications because of reduced pathogenicity compared to other viral vectors, episomal localization, and stable transgene expression. Improving the transduction efficiency of AAV particles for MSC cells would be of great benefit to diseases and disorders relevant to MSC-based therapies, including autoimmune disorders, inflammatory bowel disease (IBD), and diabetes.

Improving the transduction efficiency of AAV particles for brain cells, such as neurons, would be of great benefit to treating neurological diseases and disorders.

The tissue tropism and transduction efficiency of AAV particles is determined by the nature of amino acid residues exposed at the surface of the capsid (Wu et al., J Virol. 2006, 80(22):11393-7, herein incorporated by reference). Therefore, manipulating the amino acids of the capsid proteins provides an opportunity to fine-tune the tissue tropism of the particle and also improve transduction efficiency. However, certain manipulations, e.g., substitutions of amino acids, of the capsid protein can cause it to mis-fold or not form a capsid at all. To circumvent issues of protein mis-folding and capsid mis-forming, the recombinant AAV5 (rAAV5) variant proteins and viral particles disclosed herein were identified from a variant rAAV5 capsid library that was built by making substitutions in only the variable loops of the capsid protein. Herein, “variable loops” are also referred to as “variable regions” (VRs). As in other AAV serotypes, AAV5 has 9 variable regions, numbered from VR I to VR IX. In addition to these variable regions, additional surface loop regions have been identified that uniquely differ in AAV5 compared to other serotypes. These capsid conformational differences likely confer serotype-specific functionality involving receptor attachment, tissue transduction, and capsid assembly phenotype. In contrast to the variability observed on the exterior surface of the AAV5 capsid, the internal surface topology and the volume it encloses are highly conserved compared to those of, e.g., AAV2 and AAV4. The crystal structure of AAV is described in Govindasamy et al., J. Virol. 87(20):11187-89 (2013), which is incorporated by reference herein.

AAV particles of serotype 5 have shown fairly high levels of transduction in the central nervous system (CNS) following administration to the cerebrospinal fluid of non-human primate subjects, e.g., through intracisternal (intra-cisterna magna), intrathecal, and intracerebro-ventricular (ICV) routes of administration. See Kondratov et al., Mol. Ther. 29(9) (2021), which is incorporated by reference herein. AAV5 also showed good CNS transduction and exceptional intercellular trafficking efficiencies following intraparenchymal administration. For instance, AAV5-mediated gene therapies are currently under investigation as a therapeutic for delivery of clotting factor VIII and factor IX to subjects with hemophilia A.

Studies have indicated that AAV5 is more efficient than AAV2 at transducing neuronal and lung tissues. These observations have fueled efforts to develop AAV5 as a vector for treatment of genetic diseases of the brain and lungs. See, e.g., Lin et al. 2011. Mol. Genet. Metab. 103:367-377, which is incorporated by reference herein. Recent studies have indicated that AAV5 vectors show high transduction efficiency in neural stem cells and precursor cells in rodents (see Sehara et al., Human Gene Therapy (2021)) and long-term transduction in human-derived adipose stem cells (see Sharma et al. Hum Gene Ther Methods. 27(6): 219-227 (2016)).

In some aspects, methods and compositions of the application provide novel rAAV variants having high transduction efficiencies (e.g., similar or higher than wild type AAV5) for CNS cells and tissues. In some embodiments, rAAV5 variants are useful for targeting the forebrain, the midbrain, and/or the pons/medulla. In some embodiments, rAAV5 variants with improved transduction efficiency are useful for targeting and/or efficient delivery of gene(s) of interest to one or more regions of the brain. In some embodiments, rAAV5 variants can be useful for delivering gene(s) of interest to one or more regions of the brain even if their transduction efficiency is similar to or lower than that of wild type AAV5. For example, rAAV5 variants may have different immunological profiles and be useful to avoid a host immune response (e.g., in the context of redosing, for example when a second or additional dose of a rAAV is administered to a patient or subject, and/or if a patient or subject was previously exposed to AAV5).

In several aspects of the present disclosure, modified rAAV5 capsids are provided. In some embodiments, the transduction efficiencies in a mammalian cell (such as a human MSC) of any of the disclosed rAAV5 variants is higher than that of a corresponding wild-type AAV5 capsid. Accordingly, in some embodiments, these modified capsids possess a transduction efficiency that is at least 2-fold, at least about 4-fold, at least about 6-fold, at least about 8-fold, at least about 10-fold, or at least about 12-fold or higher in a selected mammalian host cell than that of a virion that comprises a corresponding, unmodified rAAV capsid. In certain embodiments, the transduction efficiency of the rAAV capsids provided herein will be at least about 15-fold higher, at least about 20-fold higher, at least about 25-fold higher, at least about 30-fold higher, or at least about 40, 45, or 50-fold or more greater than that of a virion that comprises a corresponding wild-type capsid.

In some aspects of the present disclosure, the transduction efficiency of any of the disclosed modified rAAV capsids is higher than that of another modified AAV5 capsid, such as AAV5-3xY/F (“AAV5 triple mutant”). In some embodiments, these modified capsids possess a transduction efficiency in a mammalian cell that is at least 2-fold, at least about 4-fold, at least about 6-fold, at least about 8-fold, at least about 10-fold, or at least about 12-fold or higher in a selected mammalian host cell than that of a virion that comprises another modified AAV5 capsid. In some embodiments, these modified capsids possess a transduction efficiency in a mammalian cell that is 2-fold or about 2-fold higher in a selected mammalian host cell than that of a virion that comprises another modified AAV5 capsid. In some embodiments, these modified capsids possess a transduction efficiency in a mammalian cell that is 2.5-fold or about 2.5-fold higher in a selected mammalian host cell than that of a virion that comprises another modified AAV5 capsid. In certain embodiments, the transduction efficiency of the disclosed modified rAAV capsids provided herein will be at least about 15-fold higher, at least about 20-fold higher, at least about 25-fold higher, at least about 30-fold higher, or at least about 40, 45, or 50-fold or more greater than that of a virion that comprises an AAV5 capsid mutant.

In some embodiments, the transduction efficiency of any of the disclosed rAAV5 variants is higher than that of the corresponding wild-type AAV5 capsid by about 15%, a 30%, a 50%, a 100%, a 200%, a 300%, a 400%, a 500%, a 750%, or 1000%, in a host cell. In some embodiments, the transduction efficiency of any of the disclosed rAAV5 mutants will be higher than that of another AAV5 mutant by about 15%, a 30%, a 50%, a 100%, a 200%, a 300%, a 400%, a 500%, a 750%, or 1000%, in a host cell.

Accordingly, provided herein are methods of transducing a host cell with a transgene of interest, the method comprising providing to a host cell a recombinant AAV particle containing any of the disclosed AAV capsid variants, or a composition thereof. In certain embodiments, the method provides about a 15%, a 30%, a 50%, a 100%, a 200%, a 300%, a 400%, a 500%, a 750%, or a 1000% increase in transduction of the transgene of interest in the host cell, relative to a wild-type recombinant AAV5 particle. The host cell may be an MSC. The host cell may be a neuron or glial cell. In certain embodiments, provided herein are methods of transducing an MSC cell (such as a human MSC cell) with a transgene of interest, the method comprising providing to the MSC a recombinant AAV particle containing any of the disclosed AAV capsid variants, or a composition thereof. In some embodiments, provided herein are methods of transducing a neuron (such as a human or murine neuron) with a transgene of interest, the method comprising providing to the neuron or glial cell a recombinant AAV particle containing any of the disclosed AAV capsid variants, or a composition thereof.

In some embodiments, relative (or differential) transduction efficiency is evaluated in vivo by measuring the differential expression of a protein encoded in the rAAV vector (which indicates the degree of transduction of that protein) of an administered virion in a sample obtained from subjects that had been administered the virions under comparison. In other embodiments, transduction efficiency is evaluated in vitro by administering to one or more cells (e.g., human cells) the virions and measuring the differential percent of transduction (i.e., % expression of encoded protein) by flow cytometry between samples. Cells cultures may be adherent or spheroid for any such evaluation. Transduction of cells may occur at any suitable MOI. In some embodiments, evaluation is performed by transducing cells at an MOI selected from between 1×10⁴ and 2×10⁸. In some embodiments, evaluation is performed by transducing cells at an MOI selected from 1×10⁴, 5×10⁴, 1×10⁵, 2.5×10⁵ and 5×10⁵. The encoded protein may be a reporter protein (e.g., a fluorescent protein) or a therapeutic protein.

The virions disclosed herein (e.g., AAV5-DK, AAV-GK, AAV-FDA, or AAV-SAG virions) demonstrate reduced seroreactivity relative to a wild-type AAV5 capsid, or relative to another AAV5 capsid mutant, such as AAV5-3xY/F. In some embodiments, the disclosed virions possess enhanced ability to evade neutralizing antibodies (NAb) of host cells in vivo, e.g., in a subject, such as a primate (e.g., a human or non-human primate).

Reduced seroreactivity and evasion of NAb in subjects may be measured by any method known in the art. In some embodiments, the degree of reduced seroreactivity and/or evasion of NAb is evaluated in vivo in human tissue by measuring the differential expression of a protein encoded in the rAAV vector (which indicates the degree of transduction of that protein) of an administered virion in a sample obtained from a subject that had been administered the virions.

In other embodiments, degree of reduced seroreactivity and/or evasion of NAb is evaluated in vitro by pre-incubating an rAAV virion encoding a protein with pooled IVIg, transducing one or more cells (e.g., human cells) with the pre-incubated virions, and measuring the differential percent of transduction (i.e., % expression of encoded protein) by flow cytometry between samples. In some embodiments, reduced seroreactivity and/or evasion of NAb is evaluated in vitro by pre-incubating an rAAV virion encoding a protein with serum samples from healthy subjects (e.g., about 50 or 100 human subjects), transducing one or more cells (e.g., human cells) with the pre-incubated virions, and measuring the percent of transduced cells (i.e., % of cells expressing encoded protein) by flow cytometry. In addition to percent of transduced cells, differential transduction between samples may be measured by fluorescence (e.g., firefly luciferase or FLuc activity) or mean fluorescence intensity (mFI). Transduction of cells may occur at any suitable MOI. In some embodiments, evaluation is performed by transducing cells at an MOI selected from between 1×10⁴ and 2×10⁸. In some embodiments, evaluation is performed by transducing cells at an MOI selected from 1×10⁴, 5×10⁴, 1×10⁵, 2.5×10⁵ and 5×10⁵. The encoded protein may be a reporter protein (e.g., a fluorescent protein) or a therapeutic protein.

In some aspects, the present disclosure provides variants of the wild-type AAV5 capsid. The wild-type AAV5 capsid VP1 region is set forth as SEQ ID NO: 1, below. In some embodiments, the variants, or modified capsids, of the present disclosure have an amino acid sequence essentially as set forth in SEQ ID NO: 1. In certain embodiments, the modified AAV capsid is truncated by 1, 2, 3, 4, 5, 6, 7, 8, 9, 10, 11, 12, 13, 14, 15, or 15-20 amino acids relative to the wild-type AAV5 VP1 sequence of SEQ ID NO: 1.

(SEQ ID NO: 1)
SFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEF
QEKLADDTSFGGNLGKAVFQAKKRVLEPFGLVEEGAKTAPTGKRIDDHF
PKRKKARTEEDSKPSTSSDAEAGPSGSQQLQIPAQPASSLGADTMSAGG
GGPLGDNNQGADGVGNASGDWHCDSTWMGDRVVTKSTRTWVLPSYNNHQ
YREIKSGSVDGSNANAYFGYSTPWGYFDFNRFHSHWSPRDWQRLINNYW
GFRPRSLRVKIFNIQVKEVTVQDSTTTIANNLTSTVQVFTDDDYQLPYV
VGNGTEGCLPAFPPQVFTLPQYGYATLNRDNTENPTERSSFFCLEYFPS
KMLRTGNNFEFTYNFEEVPFHSSFAPSQNLFKLANPLVDQYLYRFVSTN
NTGGVQFNKNLAGRYANTYKNWFPGPMGRTQGWNLGSGVNRASVSAFAT
TNRMELEGASYQVPPQPNGMTNNLQGSNTYALENTMIFNSQPANPGTTA
TYLEGNMLITSESETQPVNRVAYNVGGQMATNNQSSTTAPATGTYNLQE
IVPGSVWMERDVYLQGPIWAKIPETGAHFHPSPAMGGFGLKHPPPMMLI
KNTPVPGNITSFSDVPVSSFITQYSTGQVTVEMEWELKKENSKRWNPEI
QYTNNYNDPQFVDFAPDSTGEYRTTRPIGTRYLTRPL

Accordingly, provided herein are rAAV5 capsid proteins comprising substitutions relative to the wild-type AAV5 VP1 sequence (e.g., as set forth in SEQ ID NO: 1). In some embodiments, an amino acid substitution in any one of the variant AAV5 capsid proteins disclosed herein lies in a variable region as defined by wild-type AAV5 VP1 protein. It should be understood that any positioning of an amino acid as described herein is with respect to the sequence of the wild-type AAV5 VP1 sequence as set forth in SEQ ID NO: 1.

In some embodiments, a variant rAAV5 capsid comprises one or more amino acid substitutions in any one variable region (e.g., VRI, VRII, VRIII, VRIV, VRV, VRVI, VRVII, VRVIII or VRIX). In some embodiments, a variant rAAV5 capsid comprises one or more amino acid substitutions in more than one variable region (e.g., VRI and VRII, VRI and VRVII or VRIV, VRII).

In particular embodiments, the modified AAV capsid comprises “AAV5-DK,” which comprises the amino acid sequence set forth as SEQ ID NO: 2 (mutations are underlined), below. Elsewhere herein, the AAV5-DK variant may be referred to as “DK.”

(SEQ ID NO: 2)
MSFVDHPPDWLEEVGEGLREFLGLEAGPPKPKPNQQHQDQARGLVLPGY
NYLGPGNGLDRGEPVNRADEVAREHDISYNEQLEAGDNPYLKYNHADAE
FQEKLADDTSFGGNLGKAVFQAKKRVLEPFGLVEEGAKTAPTGKRIDDH
FPKRKKARTEEDSKPSTSSDAEAGPSGSQQLQIPAQPASSLGADTMSAG
GGGPLGDNNQGADGVGNASGDWHCDSTWMGDRVVTKSTRTWVLPSYNNH
QYREIKSGSVDGSNANAYFGYSTPWGYFDFNRFHSHWSPRDWQRLINNY
WGFRPRSLRVKIFNIQVKEVTVQDSTTTIANNLTSTVQVFTDDDYQLPY
VVGNGTEGCLPAFPPQVFTLPQYGYATLNRDNTDKPTERSSFFCLEYFP
SKMLRTGNNFEFTYNFEEVPFHSSFAPSQNLFKLANPLVDQYLYRFVST
NNTGGVQFNKNLAGRYANTYKNWFPGPMGRTQGWNLGSGVNRASVSAFA
TTNRMELEGASYQVPPQPNGMTNNLQGSNTYALENTMIFNSQPANPGTT
ATYLEGNMLITSESETQPVNRVAYNVGGQMATNNQSSTTAPATGTYNLQ
EIVPGSVWMERDVYLQGPIWAKIPETGAHFHPSPAMGGFGLKHPPPMML
IKNTPVPGNITSFSDVPVSSFITQYSTGQVTVEMEWELKKENSKRWNPE
IQYTNNYNDPQFVDFAPDSTGEYRTTRPIGTRYLTRPL

In particular embodiments, the modified AAV capsid comprises “AAV5-GK,” which comprises the amino acid sequence set forth as SEQ ID NO: 3 (mutations are underlined), below. Elsewhere herein, the AAV5-GK variant may be referred to as “GK.”

(SEQ ID NO: 3)
MSFVDHPPDWLEEVGEGLREFLGLEAGPPKPKPNQQHQDQARGLVLPGY
NYLGPGNGLDRGEPVNRADEVAREHDISYNEQLEAGDNPYLKYNHADAE
FQEKLADDTSFGGNLGKAVFQAKKRVLEPFGLVEEGAKTAPTGKRIDDH
FPKRKKARTEEDSKPSTSSDAEAGPSGSQQLQIPAQPASSLGADTMSAG
GGGPLGDNNQGADGVGNASGDWHCDSTWMGDRVVTKSTRTWVLPSYNNH
QYREIKSGSVDGSNANAYFGYSTPWGYFDFNRFHSHWSPRDWQRLINNY
WGFRPRSLRVKIFNIQVKEVTVQDSTTTIANNLTSTVQVFTDDDYQLPY
VVGNGTEGCLPAFPPQVFTLPQYGYATLNRDNTGKPTERSSFFCLEYFP
SKMLRTGNNFEFTYNFEEVPFHSSFAPSQNLFKLANPLVDQYLYRFVST
NNTGGVQFNKNLAGRYANTYKNWFPGPMGRTQGWNLGSGVNRASVSAFA
TTNRMELEGASYQVPPQPNGMTNNLQGSNTYALENTMIFNSQPANPGTT
ATYLEGNMLITSESETQPVNRVAYNVGGQMATNNQSSTTAPATGTYNLQ
EIVPGSVWMERDVYLQGPIWAKIPETGAHFHPSPAMGGFGLKHPPPMML
IKNTPVPGNITSFSDVPVSSFITQYSTGQVTVEMEWELKKENSKRWNPE
IQYTNNYNDPQFVDFAPDSTGEYRTTRPIGTRYLTRPL

In particular embodiments, the modified AAV capsid comprises “AAV5-FDA,” which comprises the amino acid sequence set forth as SEQ ID NO: 4 (mutations are underlined), below. Elsewhere herein, the AAV5-FDA variant may be referred to as “FDA.”

(SEQ ID NO: 4)
MSFVDHPPDWLEEVGEGLREFLGLEAGPPKPKPNQQHQDQARGLVLPGY
NYLGPGNGLDRGEPVNRADEVAREHDISYNEQLEAGDNPYLKYNHADAE
FQEKLADDTSFGGNLGKAVFQAKKRVLEPFGLVEEGAKTAPTGKRIDDH
FPKRKKARTEEDSKPSTSSDAEAGPSGSQQLQIPAQPASSLGADTMSAG
GGGPLGDNNQGADGVGNASGDWHCDSTWMGDRVVTKSTRTWVLPSYNNH
QYREIKSGSVDGSNANAYFGYSTPWGYFDFNRFHSHWSPRDWQRLINNY
WGFRPRSLRVKIFNIQVKEVTVQDSTTTIANNLTSTVQVFTDDDYQLPY
VVGNGTEGCLPAFPPQVFTLPQYGYATLNRDNTENPTERSSFFCLEYFP
SKMLRTGNNFEFTYNFEEVPFHSSFAPSQNLFKLANPLVDQYLFRFVST
DATGNLKFNKNLAGRYANTYKNWFPGPMGRTQGWNLGSGVNRASVSAFA
TTNRMELEGASYQVPPQPNGMTNNLQGSNTYALENTMIFNSQPANPGTT
ATYLEGNMLITSESETQPVNRVAYNVGGQMATNNQSSTTAPATGTYNLQ
EIVPGSVWMERDVYLQGPIWAKIPETGAHFHPSPAMGGFGLKHPPPMML
IKNTPVPGNITSFSDVPVSSFITQYSTGQVTVEMEWELKKENSKRWNPE
IQYTNNYNDPQFVDFAPDSTGEYRTTRPIGTRYLTRPL

In particular embodiments, the modified AAV capsid comprises “AAV5-SAG,” which comprises the amino acid sequence set forth as SEQ ID NO: 5 (mutations are underlined), below. Elsewhere herein, the AAV5-SAG variant may be referred to as “SAG.”

(SEQ ID NO: 5)
MSFVDHPPDWLEEVGEGLREFLGLEAGPPKPKPNQQHQDQARGLVLPGY
NYLGPGNGLDRGEPVNRADEVAREHDISYNEQLEAGDNPYLKYNHADAE
FQEKLADDTSFGGNLGKAVFQAKKRVLEPFGLVEEGAKTAPTGKRIDDH
FPKRKKARTEEDSKPSTSSDAEAGPSGSQQLQIPAQPASSLGADTMSAG
GGGPLGDNNQGADGVGNASGDWHCDSTWMGDRVVTKSTRTWVLPSYNNH
QYREIKSGSVDGSNANAYFGYSTPWGYFDFNRFHSHWSPRDWQRLINNY
WGFRPRSLRVKIFNIQVKEVTVQDSTTTIANNLTSTVQVFTDDDYQLPY
VVGNGTEGCLPAFPPQVFTLPQYGYATLNRDNTENPTERSSFFCLEYFP
SKMLRTGNNFEFTYNFEEVPFHSSFAPSQNLFKLANPLVDQYLYRFVST
NNTGGVQFNKNLAGRYANTYKNWFPGPMGRTQGWNLSAGGNRNYLSAKA
TTNRMELEGASYQVPPQPNGMTNNLQGSNTYALENTMIFNSQPANPGTT
ATYLEGNMLITSESETQPVNRVAYNVGGQMATNNQSSTTAPATGTYNLQ
EIVPGSVWMERDVYLQGPIWAKIPETGAHFHPSPAMGGFGLKHPPPMML
IKNTPVPGNITSFSDVPVSSFITQYSTGQVTVEMEWELKKENSKRWNPE
IQYTNNYNDPQFVDFAPDSTGEYRTTRPIGTRYLTRPL

The AAV5-FDA capsid contains the amino acid motif set forth as SEQ ID NO: 6: LFRFVSTDATGNLKF in variable region (VR) IV. Accordingly, in some embodiments, the disclosed capsid variants comprise the amino acid sequence of SEQ ID NO: 6. In some embodiments, the disclosed capsid variants comprise an 8-, 9-, 10-, 11-, or 12-amino acid fragment of the amino acid sequence of SEQ ID NO: 6.

The AAV5-SAG capsid contains the amino acid motif set forth as SEQ ID NO: 7: LSAGGNRNYLSAKA in variable region (VR) V. Accordingly, in some embodiments, the disclosed capsid variants comprise the amino acid sequence of SEQ ID NO: 7. In some embodiments, the disclosed capsid variants comprise an 8-, 9-, 10-, 11-, or 12-amino acid fragment of the amino acid sequence of SEQ ID NO: 7.

In some embodiments, the modified AAV capsid comprises an amino acid sequence that is at least 80%, at least 85%, at least 90%, at least 95%, at least 98%, or at least 99.5% identical to any one of the amino acid sequences set forth as SEQ ID NO: 2-5. In certain embodiments, the modified AAV capsid comprises any of the sequence of SEQ ID NOs: 2-5. In some embodiments, the modified AAV capsid comprises the substitutions present in any one of SEQ ID NOs: 2-5, and further one, two, three, or more than three additional substitutions relative to the VP1 sequence of SEQ ID NO: 1. In some embodiments, the modified AAV capsid consists essentially of, or consists of, the substitutions present in any one of SEQ ID NOs: 2-5, and further one, two, three, or more than three additional substitutions relative to the VP1 sequence of SEQ ID NO: 1.

In some embodiments, the modified AAV capsid comprises 1, 2, 3, 4, 5, 6, 7, 8, 9, 10, 11, 12, 13, 14, 15, or 15-20 amino acid substitutions relative to any of the amino acid sequences of any one of SEQ ID NOs: 2-5. These differences may comprise amino acids that have been inserted, deleted, or substituted relative to any one of SEQ ID NOs: 2-5. In some embodiments, the disclosed capsid rAAV variants comprise truncations at the N- or C-terminus relative to any one of SEQ ID NOs: 2-5. In some embodiments, the disclosed capsid rAAV variants comprise stretches of 15, 20, 25, 30, 35, 40, 45, 50, or more than 50 consecutive amino acids in common with any one of SEQ ID NOs: 2-5.

In some embodiments, any of the disclosed capsids comprise two or more substitutions present in SEQ ID NOs: 2 and 5, two or more substitutions present in SEQ ID NOs: 2 and 6, two or more substitutions present in SEQ ID NOs: 3 and 5, two or more substitutions present in SEQ ID NOs: 3 and 6, or two or more substitutions present in SEQ ID NOs: 5 and 6.

In some embodiments, one or more amino acid substitutions of any of SEQ ID NOs; 1-7 may be used alone or in any combination of 2 or more amino acid substitutions (e.g., 2, 3, 4, 5, 6, 7, 8, 9, 10 or more amino acid substitutions), for example in the context of an rAAV variant. In some embodiments, a capsid comprises AAV5-DK and AAV5-SAG mutations. In some embodiments, a capsid comprises AAV5-DK and AAV5-FDA mutations. In some embodiments, a capsid comprises AAV5-DK, AAV5-FDA, and AAV5-SAG mutations. In some embodiments, a capsid comprises AAV5-GK and AAV5-SAG mutations. In some embodiments, a capsid comprises AAV5-GK and AAV5-FDA mutations. In some embodiments, the capsid comprises AAV5-GK, AAV5-FDA, and AAV5-SAG mutations. In some embodiments, a capsid comprises AAV5-SAG and AAV5-FDA mutations.

In some embodiments, any of the disclosed capsid proteins are VP1 capsid proteins. In some embodiments, any of the disclosed capsid proteins are VP2 capsid proteins. In some embodiments, any of the disclosed capsid proteins are VP3 capsid proteins.

In some aspects, the present disclosure provides novel infectious rAAV virions and viral particles, as well as expression constructs that encode novel AAV virions. The present disclosure further provides novel nucleic molecules encoding one or more selected diagnostic and/or therapeutic agents for delivery to a population of mammalian cells, such as human cells, wherein the nucleic acid molecules are comprised within the disclosed rAAV virions and viral particles.

The present disclosure provides improved rAAV-based expression constructs that encode one or more therapeutic agents useful in the preparation of medicaments for the prevention, treatment, and/or amelioration of one or more diseases, disorders or conditions resulting from a deficiency in one or more cellular components. In particular, the present disclosure provides virions comprising modified capsids, as generated after screening of one or more libraries of rAAV-based genetic constructs encoding one or more selected molecules of interest, such as, for example, one or more diagnostic or therapeutic agents (including, e.g., proteins, polypeptides, peptides, antibodies, antigen binding fragments, siRNAs, RNAis, antisense oligo- and poly-nucleotides, ribozymes, and variants and/or active fragments thereof), for use in the diagnosis, prevention, treatment, and/or amelioration of symptoms of mammalian diseases, disorders, or conditions.

In some embodiments, the novel capsids of the infectious virions disclosed herein may have an improved efficiency in transducing one or more of a variety of cells, tissues and organs of interest, when compared to wild-type, unmodified capsids. The improved rAAV capsids provided herein may transduce one or more selected host cells at higher-efficiencies (and often much higher efficiencies) than conventional, wild-type rAAV capsids.

The present disclosure further provides populations and pluralities of the disclosed rAAV virions, infectious viral particles, and mammalian host cells that include one or more nucleic acid segments encoding them. The disclosed vectors and virions may be comprised within one or more diluents, buffers, physiological solutions or pharmaceutical vehicles, or formulated for administration to a mammal in one or more diagnostic, therapeutic, and/or prophylactic regimens. The disclosed viral particles, virions, and pluralities thereof may also be provided in excipient formulations that are acceptable for veterinary administration to selected livestock, exotics, domesticated animals, and companion animals (including pets and such like), as well as to non-human primates, zoological or otherwise captive specimens, and such like.

Preferably, the mammalian host cells will be human host cells, including, for example stem cells, neural cells, glial cells, blood cells, hematopoietic cells, CD34+ cells, liver cells, cancer cells, vascular cells, pancreatic cells, neural cells, ocular or retinal cells, epithelial or endothelial cells, dendritic cells, fibroblasts, or any other cell of mammalian origin, including, without limitation, stem cells (such as adult human stem cells), hepatic cells, lung cells, cardiac cells, pancreatic cells, intestinal cells, diaphragmatic cells, renal (i.e., kidney) cells, neural cells, blood cells, bone marrow cells, retinal cells or any one or more selected tissues of a mammal for which AAV-based gene therapy is contemplated. In some embodiments, the host cell is an MSC. In some embodiments, the host cell is a neuron or glial cell. In some embodiments, the host cell (such as an MSC) is derived from a mammalian subject, such as a human subject.

The present disclosure further provides compositions and formulations that include one or more of the host cells or rAAV particles (or virions) of the present disclosure together with one or more pharmaceutically acceptable buffers, diluents, or carriers. Such compositions may be included in one or more diagnostic or therapeutic kits, for diagnosing, preventing, treating or ameliorating one or more symptoms of a mammalian disease, injury, disorder, trauma or condition.

The present disclosure further includes methods for providing a mammal in need thereof with a diagnostically- or therapeutically-effective amount of a selected biological molecule, the method comprising providing to a cell, tissue or organ of a mammal in need thereof, an amount of an rAAV expression construct; and for a time effective to provide the mammal with a diagnostically- or a therapeutically-effective amount of the selected biological molecule.

The present disclosure further provides methods for diagnosing, preventing, treating, or ameliorating at least one or more symptoms of a disease, a disorder, a condition, an injury, an abnormal condition, or trauma in a mammal. In an overall and general sense, the methods include at least the step of administering to a mammal in need thereof one or more of the disclosed rAAV constructs, in an amount and for a time sufficient to diagnose, prevent, treat or ameliorate the one or more symptoms of the disease, disorder, condition, injury, abnormal condition, or trauma in the mammal.

The present disclosure also provides methods of transducing a population of mammalian cells. In an overall and general sense, the methods include at least the step of introducing into one or more cells of the population, a composition that comprises an effective amount of one or more of the rAAV virions disclosed herein.

In other aspects, the present disclosure provides compositions, as well as therapeutic and/or diagnostic kits that include one or more of the disclosed AAV compositions, formulated with one or more additional ingredients, or prepared with one or more instructions for their use.

In some aspects, the present disclosure provides methods for using the disclosed improved rAAV virions in a variety of ways, including, for example, ex situ, ex vivo, in vitro and in vivo applications, methodologies, diagnostic procedures, and/or gene therapy regimens.

In one aspect, the present disclosure provides compositions comprising AAV virions, viral particles, and pharmaceutical formulations thereof, useful in methods for delivering genetic material encoding one or more beneficial or therapeutic product(s) to mammalian cells and tissues. In particular, the compositions and methods of the present disclosure provide a significant advancement in the art through their use in the treatment, prevention, and/or amelioration of symptoms of one or more mammalian diseases. It is contemplated that human gene therapy will particularly benefit from the present teachings by providing new and improved viral vector constructs for use in the treatment of a number of diverse diseases, disorders, and conditions.

Further contemplated herein are variant rAAV capsid proteins of serotypes other than serotype 5. In some embodiments, any one of the amino acid substitutions described herein are in a variable region of the capsid protein of a serotype other than serotype 5 that is homologous to the variable region of AAV5. In some embodiments, a variant rAAV capsid protein of a serotype other than serotype 5 is of any serotype other than AAV5 (e.g., 1, 2, 3, 4, 5, 6, 7, 8, 9, 10, 11, 12 or 13). In such embodiments, provided herein are variant rAAV capsids containing mutations or substitutions that correspond to the substitutions set forth in any of the disclosed rAAV5 mutants, such as any one of SEQ ID NOs: 2-5.

### Library Design And Construction

In another aspect, the present disclosure concerns libraries of rAAV capsid variants that demonstrate improved properties useful in the delivery of one or more therapeutic agents to selected mammalian cells, and particularly for use in the prevention, treatment, and/or amelioration of one or more disorders in a mammal into which the vector construct may be introduced. In some embodiments, the disclosed libraries comprise rAAV5 capsid variants.

Comparison of the AAV VP3 structure among various serotypes has revealed highly homologous sequences interspersed with more evolutionary divergent areas. These amino acid stretches are commonly designated as VRs I through IX (variable regions I-IX; also known as “loops”). VRs are localized at the surface of the assembled capsid and are assumed to be responsible for the capsid interaction with cell surface receptors and other host factors. Because of their location, VRs are also predicted to be less critical for capsid assembly. Therefore, the guiding principle of the library's design was to modify only surface VRs while keeping the backbone sequence unchanged to maintain the integrity of the assembling scaffold. All candidate positions for mutagenesis in the AAV5 background, were selected from the alignment of known variants, which can be evaluated on a three dimensional model of the AAV5 capsid. AAV5 wildtype VR-II, VR-III and VR-IX and non-variable regions of VP3 were incorporated in the plasmid library. The wild-type AAV5 sequence is set forth in SEQ ID NO: 1.

The amino acid substitutions in the wild-type AAV5 capsid proteins disclosed herein may be epistatic, i.e. that they interact with one another, e.g., synergistically. The disclosed substitutions may be grouped into motifs of substitutions. In designing the disclosed library, motifs were introduced to the capsids simultaneously and stochastically, rather than once at a time. The substitutions in each capsid variant were determined to be epistatic and act synergistically on capsid binding and transduction behavior. These motifs confer unexpectedly enhanced transduction efficiencies in, for instance, mammalian neurons.

### Tissue-Specific or Cell-Specific Virions

The master library may be used to select virions having capsids containing degenerate or otherwise modified Cap protein (i.e., Cap protein that differs from wildtype serotype 5 one or more amino acid position(s)) that are targeted to particular tissue or cell types. For example, virions made according to the present disclosure include those that exhibit a new tropism, e.g., those capable of infecting cells normally non-permissive to AAV infection in general or at least non-permissive to AAV5 infection, as well as those that exhibit an increased or decreased ability to infect a particular cell or tissue type. As another example, virions made according to the present disclosure include those that lack the ability to infect cells normally permissive to AAV infection in general or at least normally permissive to AAV5 infection. To select for virions having a particular cell- or tissue-specific tropism, a packaged master library is introduced into a target cell. Preferably, the target cell is also infected with a helper virus (e.g., adenovirus, or Ad). The target cell is cultured under conditions that allow for the production of virions, resulting in a population of virions that are harvested from the target cell. This population of virions has been selected for having a tropism for that target cell.

As controls in a typical experiment in which virions having a particular tropism are selected, cells in different flasks or dishes may be simultaneously infected with WT AAV5 or rAAV using the same conditions as used for the library. After a suitable time post-infection, cells may be harvested, washed and the virions purified using a suitable purification method. See Gao et al., Hum. Gene Ther. 9:2353-62, 1998; U.S. Pat. No. 6,146,874; and Zolotukhin et al., Gene Ther. 6:973-85, 1999, each of which are incorporated herein by reference. AAV and helper virions (e.g., Ad) from each infection may be titered, e.g., by real-time PCR, and the AAV virions may then be further propagated, resulting in a stock of selected virions.

Once the selected population of virions having a desired tropism is isolated, nucleic acid from the virions is isolated and the sequence of the nucleotide sequence encoding the at least one AAV Cap protein is determined. Virions constructed and selected according to the present disclosure (e.g., virions comprising AAV5-DK) that can specifically target diseased cells or tissues over non-diseased cells or tissues are particularly useful.

Alternatively, tissue- or cell-specific virions may be selected using an in vivo approach. For example, mice (or other suitable host) may be injected with a suitable amount of viral preparation (e.g., 1×10¹⁰ to 1×10¹¹ vector genomes (vg) in the case of mice) via the tail vein. More than one round of selection may be performed by injecting original master library for the first round and target-enriched libraries in subsequent rounds. Hosts are euthanized after an incubation period (3 to 4 days for mice), and episomal DNA is purified from the target cells or tissue and used as a template to amplify capsid DNA sequences. Target-enriched libraries may then be generated, purified and quantified. After several rounds of selection, amplified capsid DNA may be inserted into an appropriate vector for cloning and random clones may be analyzed by sequencing.

### Expression Constructs

In some aspects, the present disclosure provides polynucleotides, or expression constructs, that encode one or more of the capsids as described herein (i.e., one or more of the disclosed AAV5 capsid variants). The polynucleotide may be comprised within a plasmid. These polynucleotides (and/or plasmids) may comprise one or more nucleotide substitutions to the nucleic acid sequence that encodes a wild-type AAV5 capsid, e.g., one or more nucleotide substitutions in one or more capsid variable region-encoding sequences. These polynucleotides (and/or plasmids) may comprise two or more nucleotide substitutions in a capsid variable region-encoding sequence such that the polynucleotide encodes the amino acid sequence of any one of SEQ ID NOs 2-7.

In some embodiments, provided herein is a polynucleotide that encodes the DK variant. In some embodiments, provided herein is a polynucleotide that encodes the GK variant. In some embodiments, provided herein is a polynucleotide that encodes the FDA variant. In some embodiments, provided herein is a polynucleotide that encodes the SAG variant.

In some aspects, the present disclosure provides rAAV nucleic acid vectors, or expression constructs or rAAV genomes, that comprise one or more transgenes comprising a sequence encoding a protein or polypeptide of interest operably linked to a promoter, wherein the one or more transgenes are flanked on each side with an ITR sequence. In some embodiments, the one or more transgenes are therapeutic transgenes. In some embodiments, the nucleic acid vector further comprises a region encoding a Rep protein as described herein, either contained within the region flanked by ITRs or outside the region or nucleic acid) operably linked to a promoter, wherein the one or more nucleic acid regions. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV5. In other embodiments, the ITR sequences of the first serotype are derived from AAV1, AAV5, AAV6, AAV7, AAV8, AAV9 or AAV10. In some embodiments, the ITR sequences are of the same serotype as the capsid (e.g., AAV5 ITR sequences and AAV5 capsid, etc.).

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al. Proc Natl Acad Sci USA. 1996; 93(24):14082-7; and Curtis A. Machida, Methods in Molecular Medicine™ Viral Vectors for Gene Therapy Methods and Protocols. 10.1385/1-59259-304-6:201 Humana Press Inc. 2003: Chapter 10, Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In other aspects, the present disclosure provides rAAV nucleic acid vectors that comprise a nucleic acid segment that further comprises a promoter, an enhancer, a post-transcriptional regulatory sequence, a polyadenylation signal, or any combination thereof, operably linked to the nucleic acid segment that encodes a transgene of interest. In certain embodiments, the promoter is a heterologous promoter, a tissue-specific promoter, a cell-specific promoter, a constitutive promoter, an inducible promoter, or any combination thereof. In some embodiments, the expression constructs of the present disclosure further include at least promoter capable of expressing, or directed to primarily express, the nucleic acid segment in a suitable host cell (e.g., an MSC cell) comprising the vector.

In certain embodiments, nucleic acid segments cloned into one or more of the novel rAAV nucleic acid vectors described herein will preferably express or encode one or more therapeutic transgenes of interest. Such transgenes of interest may encode one or more therapeutic agents, which may be selected from polypeptides, peptides, ribozymes, peptide nucleic acids, siRNAs, RNAis, antisense oligonucleotides, antisense polynucleotides, antibodies, antigen binding fragments, or any combination thereof.

Therapeutic agents useful in the disclosed vectors may include one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

In exemplary embodiments, the rAAV nucleic acid vectors obtained by the disclosed methods may encode at least one diagnostic or therapeutic protein or polypeptide selected from the group consisting of a molecular marker, an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphorin,, a semaphorin receptor, a serotonin transport protein, a serotonin uptake inhibitor, a serotonin receptor, a serpin, a serpin receptor, a tumor suppressor, and any combination thereof.

In certain applications, the rAAV nucleic acid vectors of the present disclosure may comprise one or more nucleic acid segments that encode a polypeptide selected from the group consisting of BDNF, CNTF, CSF, EGF, FGF, G-SCF, GM-CSF, gonadotropin, IFN, IFG-1, M-CSF, NGF, PDGF, PEDF, TGF, TGF-B2, TNF, VEGF, prolactin, somatotropin, XIAP1, IL-1, IL-2, IL-3, IL-4, IL-5, IL-6, IL-7, IL-8, IL-9, IL-10, IL-10(I87A), viral IL-10, IL-11, IL-12, IL-13, IL-14, IL-15, IL-16, IL-17, IL-18, and any combination thereof.

The rAAV nucleic acid vectors of the present disclosure may optionally further include one or more enhancer sequences that are each operably linked to the nucleic acid segment. Exemplary enhancer sequences include, but are not limited to, one or more selected from the group consisting of a CMV enhancer, a synthetic enhancer, a liver-specific enhancer, a stem cell-specific enhancer, a vascular-specific enhancer, a brain-specific enhancer, a neural cell-specific enhancer, a lung-specific enhancer, a muscle-specific enhancer, a kidney-specific enhancer, a pancreas-specific enhancer, retinal-specific enhancer and an islet cell-specific enhancer.

Exemplary promoters useful in the practice of the present disclosure include, without limitation, one or more heterologous, tissue-specific, constitutive or inducible promoters, including, for example, but not limited to, a promoter selected from the group consisting of a CMV promoter, a β-actin promoter, an insulin promoter, an enolase promoter, a BDNF promoter, an NGF promoter, an EGF promoter, a growth factor promoter, an axon-specific promoter, a dendrite-specific promoter, a brain-specific promoter, a hippocampal-specific promoter, a kidney-specific promoter, a retinal-specific promoter, an elafin promoter, a cytokine promoter, an interferon promoter, a growth factor promoter, an α₁-antitrypsin promoter, a brain cell-specific promoter, a neural cell-specific promoter, a central nervous system cell-specific promoter, a peripheral nervous system cell-specific promoter, an interleukin promoter, a serpin promoter, a hybrid CMV promoter, a hybrid β-actin promoter, an EF1 promoter, a U1a promoter, a U1b promoter, a Tet-inducible promoter, a VP16-LexA promoter, or any combination thereof. In exemplary embodiments, the promoter may include a mammalian or avian β-actin promoter.

The vector-encoding nucleic acid segments may also further include one or more post-transcriptional regulatory sequences or one or more polyadenylation signals, including, for example, but not limited to, a woodchuck hepatitis virus post-transcription regulatory element (WPRE), a polyadenylation signal sequence, or any combination thereof.

In some aspects, the present disclosure concerns genetically-modified rAAV nucleic acid vectors exhibiting improved transduction efficiencies that include at least a first nucleic acid segment that encodes one or more therapeutic agents that alter, inhibit, reduce, prevent, eliminate, or impair the activity of one or more endogenous biological processes in the cell. In particular embodiments, such therapeutic agents may be those that selectively inhibit or reduce the effects of one or more metabolic processes, conditions, disorders, or diseases. In certain embodiments, the defect may be caused by injury or trauma to the mammal for which treatment is desired. In other embodiments, the defect may be caused the over-expression of an endogenous biological compound, while in other embodiments still; the defect may be caused by the under-expression or even lack of one or more endogenous biological compounds.

The rAAV nucleic acid vectors of the present disclosure may also further optionally include a second distinct nucleic acid segment that comprises, consists essentially of, or consists of, one or more enhancers, one or more regulatory elements, one or more transcriptional elements, or any combination thereof, that alter, improve, regulate, and/or affect the transcription of the transgene of interest expressed by the modified rAAV vectors.

For example, the rAAV nucleic acid vectors of the present disclosure may further include a second nucleic acid segment that comprises, consists essentially of, or consists of, a CMV enhancer, a synthetic enhancer, a cell-specific enhancer, a tissue-specific enhancer, or any combination thereof. The second nucleic acid segment may also further comprise, consist essentially of, or consist of, one or more intron sequences, one or more regulatory elements, or any combination thereof.

The vectors of the present disclosure may also optionally further include a polynucleotide that comprises, consists essentially of, or consists of, one or more polylinkers, restriction sites, and/or multiple cloning region(s) to facilitate insertion (cloning) of one or more selected genetic elements, genes of interest, or therapeutic or diagnostic constructs into the rAAV construct at a selected site within the construct.

The disclosed nucleic acid vectors may be self-complementary (i.e., scrAAV nucleic acid vectors). In other embodiments, the vectors may be single-stranded.

The expression constructs and nucleic acid vectors of the present disclosure may be prepared in a variety of compositions, and may also be formulated in appropriate pharmaceutical vehicles for administration to human or animal subjects.

### Host Cells

In some aspects, the present disclosure provides host cells that comprise at least one or more of the disclosed virus particles or virions (e.g., virions comprising AAV5-DK, AAV-GK, AAV-FDA, or AAV-SAG), or one or more of the disclosed rAAV expression constructs. Such host cells are particularly mammalian host cells, with human host cells being particularly preferred, and may be either isolated, in cell or tissue culture. In the case of genetically modified animal models, the transduced host cells may even be comprised within the body of a non-human animal itself. In some embodiments, the host cells comprise humanized host cells. In particular embodiments, the host cells comprise humanized hepatocytes.

In some embodiments, provided are host cells that contain a polynucleotide that encodes one or more of the variant AAV5 capsid proteins as described herein. The host cells may contain a plasmid containing any of these polynucleotides. Provided are host cells that contain polynucleotides that encode the amino acid sequence of any one of SEQ ID NOs 2-7.

Examples of suitable host cells include MSC cells. Additional examples of host cells include neurons and glial cells. In some embodiments, the host cell (such as an MSC, neuron, or glial cell) is derived from a mammalian subject, such as a human subject. Accordingly, provided herein are MSC cells, neurons and glial cells comprising any of the disclosed virions. Further provided herein are MSC cells, neurons and glial cells comprising any of the disclosed polynucleotides encoding any of the disclosed capsid proteins.

As described above, the exogenous polynucleotide will preferably encode one or more proteins, polypeptides, peptides, ribozymes, or antisense oligonucleotides, or a combination of these. The exogenous polynucleotide may encode two or more such molecules, or a plurality of such molecules as may be desired. When combinational gene therapies are desired, two or more different molecules may be produced from a single rAAV expression construct, or alternatively, a selected host cell may be transfected with two or more unique rAAV expression constructs, each of which will provide unique transgenes encoding at least two different such molecules.

Use of rAAV Virions In Prophylaxis, Diagnosis, Or Therapy

The present disclosure also provides for uses of the compositions disclosed herein as a medicament, or in the manufacture of a medicament, for treating, preventing or ameliorating the symptoms of a disease, disorder, condition, injury or trauma, including, but not limited to, the treatment, prevention, and/or prophylaxis of a disease, disorder or condition, and/or the amelioration of one or more symptoms of such a disease, disorder or condition.

In particular embodiments, the disease, disorder or condition is selected from autoimmune disorders, inflammatory bowel disease (IBD), type 1 and type 2 diabetes, arthritis, ischemia-reperfusion injury, and cancers. In some embodiments, the disease or disorder is a cancer of the brain or CNS. In some embodiments, the disease or disorder is an autoimmune disease of the CNS.

In certain embodiments, the creation of recombinant non-human host cells, humanized host cells, and/or isolated recombinant human host cells that comprise one or more of the disclosed rAAV virions (e.g., virions comprising DK, GK, FDA, or SAG) is also contemplated to be useful for a variety of diagnostic, and laboratory protocols, including, for example, means for the production of large-scale quantities of the virions described herein. Such virus production methods may comprise improvements over existing methodologies including in particular, those that require very high titers of the viral stocks in order to be useful as a gene therapy tool. The inventors contemplate that one very significant advantage of the present methods will be the ability to utilize lower titers of viral particles in mammalian transduction protocols, yet still retain transfection rates at a suitable level.

The present disclosure provides methods of transducing a mesenchymal stem cell with a transgene of interest, the method comprising providing to the mesenchymal stem cell any of the variant recombinant AAV particles of the disclosure. In some embodiments, the MSC is a human MSC. In some embodiments, the MSC is derived from a subject suffering from a disease or disorder.

Additional aspects of the present disclosure concern methods of use of the disclosed virions, expression constructs, compositions, and host cells in the preparation of medicaments for diagnosing, preventing, treating or ameliorating at least one or more symptoms of a disease, a condition, a disorder, an abnormal condition, a deficiency, injury, or trauma in an animal, and in particular, in a vertebrate mammal, e.g., autoimmune disorders, inflammatory bowel disease (IBD), type 1 and type 2 diabetes, arthritis, ischemia-reperfusion injury, and cancers. Such methods generally involve administration to a mammal in need thereof, one or more of the disclosed virions, host cells, compositions, or pluralities thereof, in an amount and for a time sufficient to diagnose, prevent, treat, or lessen one or more symptoms of such a disease, condition, disorder, abnormal condition, deficiency, injury, or trauma in the affected animal. The methods may also encompass prophylactic treatment of animals suspected of having such conditions, or administration of such compositions to those animals at risk for developing such conditions either following diagnosis, or prior to the onset of symptoms.

The present disclosure also provides a method for treating or ameliorating the symptoms of such a disease, injury, disorder, or condition in a mammal. Such methods generally involve at least the step of administering to a mammal in need thereof, one or more of the rAAV virions as disclosed herein, in an amount and for a time sufficient to treat or ameliorate the symptoms of such a disease, injury, disorder, or condition in the mammal. Such treatment regimens are particularly contemplated in human therapy, via administration of one or more compositions either intramuscularly, intravenously, subcutaneously, intrathecally, intraperitoneally, intracisternally, intracerebro-ventricularly (ICV), intraparenchymally, or by direct injection into an organ or a tissue of the mammal under care. AAV particles of serotype 5 have shown fairly high levels of transduction in the CNS following administration through intracisternal (intra-cisterna magna), intrathecal, and ICV routes of administration.

The present disclosure also provides a method for providing to a mammal in need thereof, a therapeutically-effective amount of an rAAV composition of the present disclosure, in an amount, and for a time effective to provide the patient with a therapeutically-effective amount of the desired therapeutic agent(s) encoded by one or more nucleic acid segments comprised within the rAAV virion, e.g., a virion comprising DK, GK, FDA, or SAG. Exemplary therapeutic agents include, but are not limited to, a polypeptide, a peptide, an antibody, an antigen-binding fragment, a ribozyme, a peptide nucleic acid, an siRNA, an RNAi, an antisense oligonucleotide, an antisense polynucleotide, or a combination thereof.

Because the rAAV capsid variants of the disclosure possess enhanced ability to reduce seroreactivity and evade neutralizing antibodies, the compositions and methods provided herein facilitate the re-dosing or re-administration of an rAAV5 particle comprising any of the disclosed capsid variants to a subject who has been administered an rAAV particle previously, e.g., as part of a therapeutic regimen. This reduced seroreactivity likewise facilitates the first administration of an rAAV particle to a subject who had a previous exposure to rAAVs naively, or outside of the context of a therapeutic regimen. In some embodiments, the subject is human. In some embodiments, the previously administered rAAV particle is an rAAV5 particle. In some embodiments, the previously administered particle is of a serotype other than 5.

Accordingly, the present disclosure provides re-dosing regimens of rAAV. In some aspects of the disclosure, methods of re-administration of rAAV particles (or virions) are provided. Such methods may comprise a first administration, followed by a subsequent (or second) administration of an rAAV particle comprising any of the disclosed capsid variants. In some embodiments, such methods comprise re-administering the recombinant AAV particle or a composition comprising such a particle to the subject, e.g., a human subject in need thereof who has previously been administered the recombinant AAV particle or the composition.

### Pharmaceutical Compositions and Kits

In further aspects, the present disclosure provides compositions comprising one or more of the disclosed rAAV virions (e.g., virions comprising DK, GK, FDA, or SAG), expression constructs, infectious AAV particles, or host cells. In some embodiments, provided herein are compositions of rAAV virions that further comprise a pharmaceutically acceptable carrier for use in therapy, and for use in the manufacture of medicaments for the treatment of one or more mammalian diseases, disorders, conditions, or trauma. Such pharmaceutical compositions may optionally further comprise one or more diluents, buffers, liposomes, a lipid, a lipid complex, a microsphere or a nanoparticle.

In some embodiments, the disclosure provides pharmaceutical compositions that comprise a modified rAAV vector as disclosed herein, and further comprise a pharmaceutical excipient, and may be formulated for administration to host cell ex vivo or in situ in an animal, and particularly a human. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions, diseases or disorders as described herein.

In some embodiments, the number of rAAV particles administered to a subject may range from 10⁶ to 10¹⁴ particles. In some embodiments, rAAV particles may be administered to a subject in a dose comprising on the order ranging from 10⁶ to 10¹⁴ particles/mL or 10³ to 10¹³ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In some embodiments, from 0.01 mL to 1 mL (e.g., about 0.5 mL) or from 1 mL to 5 mL are administered to a subject. In some embodiments, rAAV particles in an amount of between 10¹¹ and 4×10¹² particles/mL are administered. In some embodiments, rAAV particles of higher than 10¹³ particles/mL are administered. In some embodiments, the number of rAAV particles administered to a subject may be on the order ranging from 10⁶ to 10¹⁴ vector genomes. In some embodiments, rAAV particles may be administered to a subject in a dose comprising on the order ranging from 10⁶ to 10¹⁴ vector genomes (vgs)/mL or 10³ to 10¹⁵ vgs/mL, or any values there between for either range, such as for example, about 10⁶, 10⁷, 10⁸, 10⁹, 10¹⁰, 10¹¹, 10¹², 10¹³, or 10¹⁴ vgs/mL. In some embodiments, from 0.01 mL to 1 mL (e.g., about 0.5 mL) or from 1 mL to 5 mL are administered to a subject. In some embodiments, a dose of between 1×10¹¹ and 2×10¹¹ vgs/ml (or between 5×10¹⁰ and 1×10¹¹ vgs/kg of subject) is administered to the subject. In some embodiments, a dose of between 1×10¹² and 4×10¹² vgs/ml (or between 5×10¹¹ to 2×10¹² vgs/kg of the subject) is administered to the subject.

In some embodiments, where a second nucleic acid vector encoding the Rep protein within a second rAAV particle is administered to a subject, the ratio of the first rAAV particle to the second rAAV particle is 1:100, 1:50, 1:40, 1:30, 1:20, 1:10, 1:5, 1:2 or 1:1. In some embodiments, the Rep protein is delivered to a subject such that target cells within the subject receive at least two Rep proteins per cell.

In some embodiments, the disclosure provides formulations of compositions disclosed herein in pharmaceutically acceptable carriers for administration to a cell or an animal, either alone or in combination with one or more other modalities of therapy, and in particular, for therapy of human cells, tissues, and diseases affecting man.

If desired, rAAV particle or preparation, Rep proteins, and nucleic acid vectors may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles or preparations, Rep proteins, and nucleic acid vectors may thus be delivered along with various other agents as required in the particular instance. Such compositions may be purified from host cells or other biological sources, or alternatively may be chemically synthesized as described herein.

The formulation of pharmaceutically acceptable carriers is well-known to those of skill in the art, as is the development of suitable dosing and treatment regimens for using the particular compositions described herein in a variety of treatment regimens, including e.g., oral, parenteral, intravenous, intranasal, intra-articular, intraparenchymal, intrathecal, intracerebro-ventricular, intracisternal, and intramuscular administration and formulation.

Typically, these formulations may contain at least about 0.1% of the therapeutic agent (e.g., rAAV particle or preparation, Rep protein, and/or nucleic acid vector) or more, although the percentage of the active ingredient(s) may, of course, be varied and may conveniently be between about 1% or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV particles or preparations, Rep proteins, and/or nucleic acid vectors in suitably formulated pharmaceutical compositions disclosed herein either subcutaneously, intraocularly, intravitreally, parenterally, subcutaneously, intravenously, intracisternally, intracerebro-ventricularly, intraparenchymally, intramuscularly, intrathecally, orally, intraperitoneally, by oral or nasal inhalation, or by direct injection to one or more cells, tissues, or organs by direct injection.

The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The pharmaceutical compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intracisternal injection.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Ed., 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations, Rep proteins, and/or nucleic acid vectors, in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Ex vivo delivery of cells (such as MSC cells or neurons) transduced with rAAV particles or preparations, and/or Rep proteins is also contemplated herein. Ex vivo gene delivery may be used to transplant rAAV-transduced host cells (e.g., MSCs) back into the host. A suitable ex vivo protocol may include several steps. For example, a segment of target tissue or an aliquot of target fluid may be harvested from the host and rAAV particles or preparations, and/or Rep proteins may be used to transduce a nucleic acid vector into the host cells in the tissue or fluid. These genetically modified cells may then be transplanted back into the host. Several approaches may be used for the reintroduction of cells into the host, including intravenous injection, intraperitoneal injection, or in situ injection into target tissue. Autologous and/or allogeneic cell transplantations of any of the disclosed host cells may be used according to the invention. In some embodiments, autologous transplantation of any of the disclosed MSC host cells is used in the treatment of a subject suffering from a genetic disease or disorder.

The amount of rAAV particle or preparation, Rep protein, or nucleic acid vector compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or preparation, Rep protein, or nucleic acid vector compositions, either over a relatively short, or a relatively prolonged period of time, as may be determined by the medical practitioner overseeing the administration of such compositions.

Toxicity and efficacy of the compositions utilized in methods of the disclosure can be determined by standard pharmaceutical procedures, using either cells in culture or experimental animals to determine the LD₅₀ (the dose lethal to 50% of the population). The dose ratio between toxicity and efficacy the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED₅₀ with little or no toxicity. The dosage may vary within this range depending upon the dosage form employed and the route of administration utilized.

The present disclosure provides compositions including one or more of the disclosed rAAV virions (e.g., virions comprising DK, GK, FDA, or SAG) comprised within a kit for diagnosing, preventing, treating or ameliorating one or more symptoms of a mammalian disease, injury, disorder, trauma or condition. Such kits may also be useful in the diagnosis, prophylaxis, and/or therapy or a human disease, and may be particularly useful in the treatment, prevention, and/or amelioration of one or more symptoms of Wilson's Disease, wet age-related macular degeneration, dry age-related macular degeneration, glaucoma, retinitis pigmentosa, diabetic retinopathy, orphan ophthalmological diseases, cancer, diabetes, autoimmune disease, kidney disease, cardiovascular disease, pancreatic disease, intestinal disease, liver disease, neurological disease, neuromuscular disorder, neuromotor deficit, neuroskeletal impairment, neurological disability, neurosensory condition, stroke, ischemia, alpha 1-antitrypsin (AAT) deficiency, Transthyretin-Related Familial Amyloid Polyneuropathy, Ornithine Transcarbamylase Deficiency, Batten's disease, Alzheimer's disease, sickle cell disease, 0-thalassemia, Huntington's disease, Parkinson's disease, skeletal disease, trauma, pulmonary disease in a human. In some embodiments, the disease or disorder is an autoimmune disorder, inflammatory bowel disease (IBD), type 1 diabetes, type 2 diabetes, arthritis, ischemia-reperfusion injury, or a cancer.

Kits comprising one or more of the disclosed rAAV virions, transduced host cells or pharmaceutical compositions comprising such vectors; and instructions for using such kits in one or more therapeutic, diagnostic, and/or prophylactic clinical embodiments are also provided by the present disclosure. Such kits may further comprise one or more reagents, restriction enzymes, peptides, therapeutics, pharmaceutical compounds, or means for delivery of the composition(s) to host cells, or to an animal (e.g., syringes, injectables, and the like). Exemplary kits include those for treating, preventing, or ameliorating the symptoms of a disease, deficiency, condition, and/or injury, or may include components for the large-scale production of the viral vectors themselves, such as for commercial sale, or for use by others, including e.g., virologists, medical professionals, and the like.

Methods of Making rAAV5 Particles

Various methods of producing rAAV particles (e.g., particles comprising AAV5-DK) and nucleic acid vectors are known (see, e.g., Zolotukhin et al. Methods 28 (2002) 158-167; and U.S. Patent Publication Nos. US 2007/0015238 and US 2012/0322861, each of which are incorporated herein by reference; and plasmids and kits available from ATCC and Cell Biolabs, Inc.). In some embodiments, a vector (e.g., a plasmid) comprising a transgene of interest may be combined with one or more helper plasmids, e.g., that contain a rep gene (e.g., encoding Rep78, Rep68, Rep52 and Rep40) and a cap gene (encoding VP1, VP2, and VP3, including a modified VP region as described herein), and transfected into a recombinant cells, called helper or producer cells, such that the nucleic acid vector is packaged or encapsidated inside the capsid and subsequently purified.

A helper cell may comprise rep and/or cap genes that encode the Rep protein and Cap proteins, respectively. Exemplary helper cells include insect cells and mammalian cells. An exemplary insect helper cell is the Sf9 cell (see, e.g., ATCC® CRL-1711™). The biological potency of AAV5-based virions in brain tissue can be substantially increased if the capsid is manufactured in Sf9 cells in which the ratio of the capsid proteins can be adjusted to incorporate higher VP1 content than VP2 and VP3 content. See Kondratov et al., Mol. Ther. 25, 2661-2675 (2017), and Mietzsch et al., Hum. Gene Ther. 26, 688-697 (2015), each of which is incorporated by reference herein. Non-limiting examples of mammalian helper cells include HEK293 cells, COS cells, HeLa cells, BHK cells, and CHO cells (see, e.g., ATCC® CRL-1573™, ATCC® CRL-1651™, ATCC® CRL-1650™, ATCC® CCL-2, ATCC® CCL-10™, or ATCC® CCL-61™). In some embodiments, the packaging is performed in vitro (e.g., outside of a living organism).

Biological potency of the disclosed AAV5 capsid mutants may be enhanced by manufacturing the rAAV particle (e.g., in an Sf9 cell) using modified Kozak sequences (translation initiation sequences) that enhance the ratio of VP1:VP2:VP3 content in a manner that favors potency. Accordingly, in some aspects, the disclosure provides nucleic acid molecules comprising a nucleotide sequence encoding a modified Kozak sequence and any of the disclosed modified AAV5 VP1, VP2, and VP3 capsid proteins. In some embodiments, the Kozak sequence comprises the initiation codon for translation of the AAV5 VP1 capsid protein and additional nucleotides upstream of the initiation codon. In some embodiments, the Kozak sequence further comprises nucleotides downstream of the initiation codon. See International Patent Publication No. WO 2017/181162, which is herein incorporated by reference.

In some embodiments, a nucleic acid vector (e.g., a plasmid) containing the transgene of interest (e.g., ATP7B) is combined with one or more helper plasmids, e.g., that contain a rep gene of a first serotype and a cap gene of the same serotype or a different serotype, and transfected into helper cells such that the rAAV particle is packaged. In some embodiments, the one or more helper plasmids include a first helper plasmid comprising a rep gene and a cap gene, and a second helper plasmid comprising one or more of the following helper genes: E1a gene, E1b gene, E4 gene, E2a gene, and VA gene. For clarity, helper genes are genes that encode helper proteins E1a, E1b, E4, E2a, and VA. Helper plasmids, and methods of making such plasmids, are known in the art and commercially available (see, e.g., pDF6, pRep, pDM, pDG, pDPlrs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adeno associated Virus Vectors, Human Gene Therapy, Vol. 9, 2745-2760; Kern, A. et al. (2003), Identification of a Heparin-Binding Motif on Adeno-Associated Virus Type 2 Capsids, J. Virol., Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188). Plasmids that encode wild-type AAV coding regions for specific serotypes are also knows. For example, pSub201 is a plasmid that comprises the coding regions of the wild-type AAV2 genome (Samulski et al. (1987), J Virology, 6:3096-3101).

Inverted terminal repeat (ITR) sequences and plasmids containing ITR sequences are known in the art and are commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al., Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman et al.; U.S. Pat. Nos. 5,139,941 and 5,962,313, all of which are incorporated herein by reference).

An exemplary Genbank reference number for wild-type AAV serotype 5 is No. NC_006152.1, which is incorporated herein by reference in its entirety.

A non-limiting method of rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes, cap genes, and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. As an example, HEK293 cells (available from ATCC®) are transfected via CaPO₄-mediated transfection, lipids or polymeric molecules such as Polyethylenimine (PEI) with the helper plasmid(s) and a plasmid containing a nucleic acid vector. The HEK293 cells are then incubated for at least 60 hours to allow for rAAV particle production. Alternatively, the HEK293 cells are transfected via methods described above with AAV-ITR containing one or more genes of interest, a helper plasmid comprising genes encoding Rep and Cap proteins, and co-infected with a helper virus. Helper viruses are viruses that allow the replication of AAV. Examples of helper virus are adenovirus (e.g., Ad5) and herpesvirus.

Alternatively, in another example, Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known in the art or described herein, e.g., by iodixanol step gradient, CsCl gradient, chromatography, or polyethylene glycol (PEG) precipitation. See US Patent Publication No. 2017/0130208, incorporated herein by reference.

In some embodiments, a baculovirus system (e.g., the OneBac system) is used for making rAAV particles. See, for example, Mietzsch, et al (Hum Gene Ther. 2014, 25(3):212-22).

Methods for large-scale production of AAV using a herpesvirus-based system are also known. See for example, Clement et al. (Hum Gene Ther. 2009, 20(8):796-806). Methods of producing exosome-associated AAV, which can be more resistant to neutralizing anti-AAV antibodies, are also known (Hudry et al., Gene Ther. 2016, 23(4):380-92; Macguire et al., Mol Ther. 2012, 20(5):960-71). Methods for producing and using pseudotyped rAAV vectors are also known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Illustrative embodiments of the present disclosure are described below. In the interest of clarity, not all features of an actual implementation are described in this specification. It will be appreciated by one of skill in the art that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

### Definitions

Unless otherwise defined, all technical terms used herein have the same meaning as commonly understood by one of ordinary skill in the art to which this present disclosure belongs. Commonly understood definitions of molecular biology terms can be found in Rieger et al., (1991) and Lewin (1994). Commonly understood definitions of virology terms can be found in Granoff and Webster (1999) and Tidona and Darai (2002).

In accordance with convention, the words “a” and “an” when used in this application, including the claims, denotes “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is acceptable for administration to the relevant animal. A “pharmaceutically acceptable carrier” is pharmaceutically acceptable for administration to a subject or patient. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

The term “e.g.,” as used herein, is used merely by way of example, without limitation intended, and should not be construed as referring only those items explicitly enumerated in the specification.

As used herein, “an effective amount” would be understood by those of ordinary skill in the art to provide a therapeutic, prophylactic, or otherwise beneficial effect against the organism, its infection, or the symptoms of the organism or its infection, or any combination thereof.

The phrase “helper function” is meant as a functional activity performed by a nucleic acid or polypeptide that is derived from a virus such as Adenovirus (Ad) or herpesvirus and that facilitates AAV replication in a host cell.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity may be determined by analyzing two or more sequences using algorithms and computer programs known in the art. Such methods may be used to assess whether a given sequence is identical or homologous to another selected sequence.

As used herein, “homologous” means, when referring to polynucleotides, sequences that have the same essential nucleotide sequence, despite arising from different origins.

Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the terms “humanize” and “humanized” refers to the action of engrafting human cells or tissues into a non-human animal, such as a mouse. The present disclosure may refer to humanized murine models and/or subjects, such as mouse models grafted with human MSCs.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

Typically, a selected sequence and the reference sequence will have at least about 80, 81, 82, 83, 84 or even 85% sequence identity, and more preferably, at least about 86, 87, 88, 89, 90, 91, 92, 93, 94, or 95% sequence identity. More preferably still, highly homologous sequences often share greater than at least about 96, 97, 98, or 99% sequence identity between the selected sequence and the reference sequence to which it was compared. The percentage of sequence identity may be calculated over the entire length of the sequences to be compared, or may be calculated by excluding small deletions or additions which total less than about 25% or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “isolated” refers to material that is substantially, or essentially, free from components that normally accompany the material as it is found in its native state. Thus, isolated polynucleotides in accordance with the present disclosure preferably do not contain materials normally associated with those polynucleotides in their natural, or in situ, environment.

As used herein, the term “kit” may be used to describe variations of the portable, self-contained enclosure that includes at least one set of components to conduct one or more of the diagnostic or therapeutic methods of the present disclosure.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

The term “library” refers to a collection of elements that differ from one another in at least one aspect. For example, a vector library is a collection of at least two vectors that differ from one another by at least one nucleotide. As another example, a “virion library” is a collection of at least two virions that differ from one another by at least one nucleotide or at least one capsid protein.

As used herein, the term “master library” refers to a pool of rAAV virions composed of chimeric rcAAV nucleic acid vectors encapsidated in cognate chimeric capsids (e.g., capsids containing a degenerate or otherwise modified Cap protein). As used herein, the term “rcAAV nucleic acid vector” refers to a replication-competent AAV-derived nucleic acid capable of DNA replication in a cell without any additional AAV genes or gene products.

When referring to a nucleic acid molecule or polypeptide, the terms “wild-type” and “native” refer to a naturally-occurring (e.g., a wild-type) nucleic acid or polypeptide. These terms refer to the fact that the described molecule may be found in nature. For example, a polypeptide or polynucleotide sequence that is present in an organism (including viruses) that may be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, the phrase “nucleic acid” means a chain of two or more nucleotides such as RNA (ribonucleic acid) and DNA (deoxyribonucleic acid). Conventional nomenclature exists in the art for polynucleotide and polypeptide structures. For example, one-letter abbreviations are widely employed to describe nucleotides: Adenine (A), Guanine (G), Cytosine (C), Thymine (T), Uracil (U), Purine, i.e. A or G (R), Pyrimidine, i.e. C or T (Y), any nucleotide (N), Weak, i.e. A or T (W), Strong, i.e. G or C(S), Amino, i.e. A or C (M), Keto, i.e. G or T (K), not A, i.e. G or C or T (B), not G, i.e. A or C or T (H), not C, i.e. A or G or T (D) and not T, i.e. A or G or C (V). In accordance with the present disclosure, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

The phrases “cap nucleic acid,” “cap gene,” and “capsid gene” as used herein mean a nucleic acid that encodes a Cap protein. Examples of cap nucleic acids include “wild-type” (WT) Cap-encoding nucleic acid sequences from AAV serotypes 1, 2, 3, 4, 5, 6, 7, 8, 9, 10, 11, 12, 13; a native form cap cDNA; a nucleic acid having sequences from which a cap cDNA can be transcribed; and/or allelic variants and homologs of the foregoing.

The terms “VR”, “VRs”, “variable region” or “variable regions” refer to amino acid stretches of capsid protein that do not have a high degree of homology between AAV variants. These amino acid stretches are commonly designated as VRs I through IX (also known as “loops”). VRs are localized at the surface of the assembled capsid and interact with host cell surface receptors and other host factors.

The phrase “pharmaceutically acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present disclosure may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids.

Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Additional conventions include: Asn or Asp (B; Asx), Gln or Glu (Z; Glx), Leu or Ile (J; Xle), Selenocysteine (U; Sec), Pyrrolysine (O; Pyl) and Unknown (X; Unk). Amino acid residues described herein are preferred to be in the “L” isomeric form. However, residues in the “D” isomeric form may be substituted for any L-amino acid residue provided the desired properties of the polypeptide are retained.

The term “promoter,” as used herein refers to a region or regions of a nucleic acid sequence that regulates transcription.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from two to about 20 amino acid residues in length, oligopeptides from about 10 to about 100 amino acid residues in length, and longer polypeptides including those of about 100 or more amino acid residues in length. Furthermore, the term is also intended to include enzymes, i.e., functional biomolecules including at least one amino acid polymer. Polypeptides and proteins of the present disclosure also include polypeptides and proteins that are or have been post-translationally modified, and include any sugar or other derivative(s) or conjugate(s) added to the backbone amino acid chain.

The term “pseudotyped” is meant a nucleic acid or genome derived from a first AAV serotype that is encapsidated (packaged) into an AAV capsid containing at least one AAV Cap protein of a second serotype differing from the first serotype. Exemplary pseudotyped AAV vectors contain AAV2 ITRs. Exemplary such vectors include AAV2/9, AVV2/8, and AAV2/1 vectors.

The term “recombinant” indicates that the material (e.g., a polynucleotide or a polypeptide) has been artificially or synthetically (non-naturally) altered by human intervention. The alteration may be performed on the material within or removed from, its natural environment or state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including a mammal such as a human primate, to which treatment with one or more of the disclosed compositions may be provided. Mammalian species that may benefit from the disclosed treatment methods include, without limitation, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. The term “host” refers to any host organism that may receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner.

As used herein, the terms “inverted terminal repeat” or “ITR” refer to a nucleic acid sequence derived from an AAV that is required in cis for replication and packaging of AAV.

As used herein, the term “transduced cell” is intended to mean a host cell whose nucleic acid complement has been altered by the introduction of one or more exogenous polynucleotides into that cell. As used herein, the term “transduction” generally describes a process of introducing an exogenous polynucleotide sequence (e.g., a viral particle, a plasmid, or a recombinant DNA or RNA molecule) into a host cell or protoplast in which the exogenous polynucleotide is incorporated into at least a first chromosome or is capable of autonomous replication within the transduced host cell. Transfection, electroporation, and “naked” nucleic acid uptake all represent examples of techniques used to transduce a host cell with one or more polynucleotides.

As used herein, the terms “treat,” “treating,” and “treatment” refer to the administration of a composition to reduce the frequency or severity of at least one sign or symptom of a disease, disorder or condition experienced by a subject. These terms embrace prophylactic administration, i.e., prior to the onset of clinical symptoms of a disease state so as to prevent any symptom, aspect or characteristic of the disease state. The disclosed compositions may be administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell. Such treating need not be absolute to be deemed medically useful. As such, the terms “treatment,” “treat,” “treated,” or “treating” may refer to therapy, or the amelioration or reduction in the extent or severity of disease, disorder or condition, of one or more symptom thereof, whether before or after onset of the disease, disorder or condition.

As used herein, the term “vector” refers to a nucleic acid molecule capable of transporting another nucleic acid to which it has been linked, e.g., a plasmid. One type of preferred vector is an episome, i.e., a nucleic acid capable of extra-chromosomal replication. Preferred vectors are those capable of autonomous replication and/or expression of nucleic acids to which they are linked. An “rAAV nucleic acid vector” is a recombinant AAV-derived nucleic acid containing at least one inverted terminal repeat (ITR) sequence.

The use of “virion” is meant to describe a virus particle that contains a nucleic acid and a protein coat (capsid). An “rAAV virion” is a virion that includes nucleic acid sequences and/or proteins derived from a rAAV expression construct.

As used herein, the term “tropism” refers to the cells and/or tissues of a host which support growth of a particular serotype of AAV. Some serotypes may have a broad tissue tropism and can infect many types of cells and tissues. Other serotypes may infect primarily a single tissue or cell type. For example, the AAV capsids of the present disclosure have a high tropism for mesenchymal stem cells.

As used herein, the term “variant” refers to a molecule (e.g., a capsid protein) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type capsid. Variants of a protein molecule, e.g., a capsid, may contain modifications to the amino acid sequence (e.g., having 1, 2, 3, 4, 5, 6, 7, 8, 9, 10, 10-15, or 15-20 amino acid substitutions) relative to the wild type protein sequence, which arise from point mutations installed into the nucleic acid sequence encoding the capsid protein. These modifications include chemical modifications as well as truncations.

By a protein (e.g., a capsid protein) comprising an amino acid sequence having at least, for example, 95% “identity” to a query amino acid sequence, it is intended that the amino sequence of the subject amino acid molecule is identical to the query sequence except that the subject amino acid molecule sequence may include up to five amino acid alterations per each 100 amino acids of the query sequence. In other words, to obtain a capsid having an amino sequence at least 95% identical to a reference (query) sequence, up to 5% of the amino acids in the subject sequence may be inserted, deleted, or substituted with another nucleotide. These alterations of the reference sequence may occur at the N- or C-terminus of the reference sequence or anywhere between those positions, interspersed either individually among amino acids in the reference sequence or in one or more contiguous groups within the reference sequence.

As a practical matter, whether any particular amino acid molecule is at least 80%, 85%, 90%, 95%, 96%, 97%, 98%, or 99% identical to, for instance, the amino acid sequence of a capsid protein, can be determined conventionally using known computer programs. A preferred method for determining the best overall match between a query sequence (e.g., a sequence of the present disclosure) and a subject sequence, also referred to as a global sequence alignment, can be determined using the FASTDB or blastn computer program based on the algorithm of Brutlag et al. (Comp. App. Biosci. 6:237-245 (1990)). In a sequence alignment the query and subject sequences are either amino acid sequence or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the N- or C-terminus, relative to the query sequence, the percent identity is corrected by calculating the number of nucleotides of the query sequence that are positioned N- or C-terminus to the query sequence, which are not matched/aligned with a corresponding subject nucleotide, as a percent of the total bases of the query sequence.

### Examples

The following examples are included to demonstrate illustrative embodiments of the present disclosure. It should be appreciated by those of ordinary skill in the art that the techniques disclosed in these examples represent techniques discovered to function well in the practice of the present disclosure, and thus can be considered to constitute preferred modes for its practice. However, those of ordinary skill in the art should, in light of the present disclosure appreciate that many changes may be made in the specific embodiments which are disclosed and still obtain a like or similar result without departing from the spirit and scope of the present disclosure.

Strategies to improve AAV capsid design include (a) rational approach of mutagenizing known capsid residues critical for binding, entry, and/or intracellular trafficking, and (b) directed evolution to rapidly introduce molecular modifications into the AAV capsids, thus manipulating both diversity and selection. Using detailed knowledge of AAV5 capsid structures, a combinatorial capsid library was derived in which only variable regions (VRs) on the surface of virion are modified.

### Example 1: AAV5 Library Generation and Characterization

The AAV icosahedral capsid is composed of three structural proteins (VP1, VP2, and VP3), of which VP2 and VP3 are N-terminal truncated versions of VP1. VP3 is the most abundant capsid subunit, comprising a major part of the capsid surface, and hence the major determinant of antigenicity and cell tropism. The structure of VP3 contains a P-barrel core, with homologous P-strands linked by 9 highly variable extended loops called variable regions (VR-I to VR-IX). Because of their surface location, VRs are predicted to be less critical for capsid assembly but essential for determining cell tropism and antigenicity. For this study, a replication competent AAV5 capsid library was generated by modifying only surface VRs while keeping the backbone sequence unchanged to maintain the integrity of the assembling scaffold.

Five lines of human mesenchymal stem cells (i.e., Mesenchymal Stromal Cells, or MSC) of low passages (P2-P3) were obtained from UF Center for Regenerative Medicine, as follows:
(1) Lipo143 ADSC;
(2) 082410 A252 ADSC;
(3) ADSC, derived from human patient 2;
(4) BM MSC line 2; and
(5) BM MSC line 1,

where “ADSC” refers to MSCs derived from adipose tissue of subjects; and “BM” refers to MSCs derived from the bone marrow. Cells were propagated for two additional passages, frozen and stored in liquid nitrogen. Aliquots of each cell line were expanded to about 3×10⁷ cells in T175 flasks.

MSC cells in each batch were infected with members of the AAV5 combinatorial library at a low multiplicity of infection (MOI) of 2.5. The following day, cells were washed twice with 1xPBS, and then infected with wild-type adenovirus 5 (Ad5) at an MOI of 5.

At day five (D5) post-AAV infection, the majority of cells developed a cytopathic effect. (A cytopathic effect refers to structural changes in host cells that are caused by viral infection, such as cell lysis.) At this time, cells in the culture medium were collected, and AAV virus was purified, individually from each cell line batch, following a standard iodixanol gradient protocol. Each AAV virus batch was titered in an iodixanol fraction, concluding a first round of directed evolution (DE). Four additional rounds of DE were performed (in total, 5 rounds for each cell line batch).

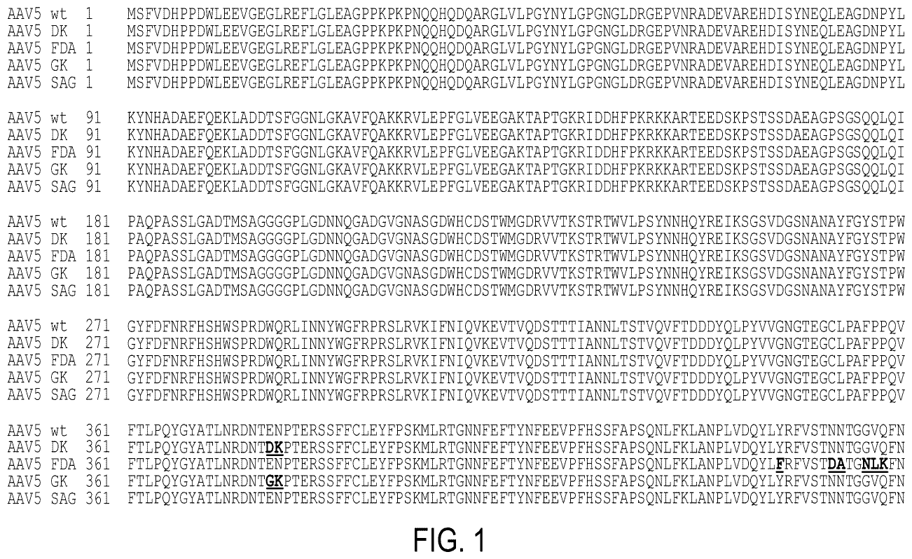

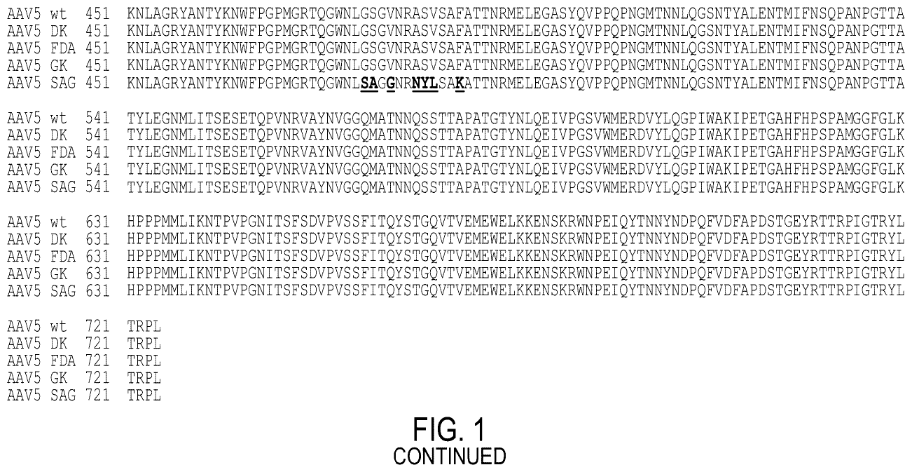

After DE Rounds 4 and 5, AAV viral DNAs were isolated, and PCR fragments of a capsid gene were subcloned into pACG2r5c helper plasmid in which a wild-type AAV5 sequence was substituted. Up to 10 individual random colonies from each DE Round, and each batch, were screened. Viral DNA plasmids were isolated from each cell batch and subjected to Sanger sequencing. A clustering analysis identified four AAV capsid variants that showed “enrichment,” i.e., exhibited increased transduction of each batch of MSC cells. These four capsids are AAV-DK (SEQ ID NO: 2), AAV-GK (SEQ ID NO: 3), AAV-FDA (SEQ ID NO: 4), and AAV-SAG (SEQ ID NO: 5), as provided in the alignment shown in FIG. 1.

Sanger sequencing was used to identify the amino acid substitutions in the capsid proteins of AAV particles identified in the directed evolution screen that conferred enhanced transduction. Several plasmids showing “enrichment” over the five rounds of DE were selected for characterization. The respective rAAV-GFP vectors were packaged using pACG2r5C incorporating selected library capsid mutants. MSCs were infected at the MOI of 10⁴, side-by side with AAV-DJ-GFP (positive control), and wt AAV5-GFP (negative control) vectors. The capsid variant AAV-DJ contains an insertion of 7 amino acids into the heparan sulfate proteoglycan binding domain of the AAV2 capsid and has high transduction efficiency in some cells of the eye, such as Muller glial cells. Like AAV5, AAV-DJ has shown high transduction efficiencies in the CNS following administration through intracisternal (intra-cisterna magna), intrathecal, and ICV routes of administration.

Subsequently, the transduction efficiencies of the four variants were evaluated in the brains of murine subjects in vivo. High transduction was observed in neurons of subjects following administration of multiple rAAV particles containing one of these four variants (data not shown).

Each of these four variants exhibited a different “enrichment factor” of the rAAV vector (or genome) associated with each variant. The enrichment factor (“%”) refers to the percentage of all nucleotide sequence reads (or “copies”) that are represented by the genome associated with a particular variant, in the particular screening round. Reads are determined through Sanger sequencing (or next-generation sequencing (NGS)) of the rAAV genomes. The higher the value of this percentage, the better the variant is suited to target and infect these particular cell types so that they integrate the genome. After a single screening round, variants generating a higher percentage of sequence reads may be selected as better (or the best) candidates. In that scenario, the % shown is equivalent to the fraction of (count of individual mutant genome reads)/(count of all mutant genome reads in the DNA sample). For example, if 10,000 copies of Variant #1 were recovered after analysis of of a total of 1000,000 copies of DNA (for that sample) were screened, then the % for Variant #1 is calculated as (10,000/1000,000)*100%, or 1%.

The fold-change values describing enrichment between two rounds of screening (e.g., rounds 4 and 5 of selection in cells) may be calculated as:

$$DNA enrichment = \frac{\% i n reads of DNA Round 45 \%}{\% of reads of in DNA Round 51 \%} = 5 times ( round 4 to 5 enrichment ) .$$

Accordingly, in some embodiments, the capsid variant of any of the disclosed rAAV particles comprises a polypeptide sequence that exhibits an enrichment factor (%) in MSC cells that is greater than 0.01, 0.05, 0.1, 0.15, 0.2, 0.25, 0.3, 0.35, 0.4, 0.45, 0.5, 1.01.5, 2.0, 2.5, 3.0, 3.5, 4.0, 4.5, 5.0, 5.5, 6.0, 6.5, 7.0, 7.5, 8.0, 8.5, 9.0, 9.50, 10.0, 11.0, 12.0, 13.0, 14.0, 15.0, 16.0, 17.0, 18.0, 19.0, 20.0, 30.0, 40.0, 50.0, 60.0, 70.0, 80.0, 90.0, or 95.0, optionally greater than 10.0, or 20.0, 30.0, 40.0, 50.0, 60.0, 70.0, 80.0, or 90.0. In particular embodiments, the capsid variants exhibit an enrichment factor of greater than 10.0, 11.0, 12.0, 13.0, 14.0, 15.0, or 20.0. For example, the capsid may comprise a sequence that comprises the substitutions of either of SEQ ID NOs: 6 or 7 relative to SEQ ID NO: 1 and that demonstrates an enrichment factor as described above.

### Example 2: Assessment of AAV5 Variants in a Mouse Model

AAV5 variants as disclosed herein were injected into mice to assess vector genome distribution in an animal model.

AAV variants were prepared to titers shown in Table 1.

TABLE 1
AAV virus variants and vector genome titers.

| AAV5-Virus | AAV5 Variant | Total vg |
|---|---|---|
| A | pAAV5-WT | 5.51E+13 |
| B | pAAV5-DK | 3.48E+13 |
| C | pAAV5-GK | 3.54E+13 |
| D | pAAV5-DK-FDA-SAG | 1.86E+13 |
| E | pAAV5-FDA | 1.50E+13 |
| F | pAAV5-SAG | 9.65E+12 |
| G | pAAV5-DK-FDA | 4.74E+13 |
| H | pAAV5-DK-SAG | 1.17E+13 |
| I | pAAV5-FDA-SAG | 2.51E+09 |

Male C57BL/6 (B16) mice were injected with the AAV variants at 8 weeks of age. Three mice were injected per variant. The route of administration (RoA) for each mouse was bilateral injection into the caudate putamen (Cpu) (basal ganglia) and injection into the right lateral ventricle (ICV, or intracerebroventricular). 3¹⁰ vector genomes total, or 1.5¹⁰ vector genomes per site (2 μl/site) were injected into the basal ganglia. 7.5¹⁰ (7.5 μl) was injected into the right ICV. Mice were injected at a rate of 0.5 μl/minute for both routes of injection.

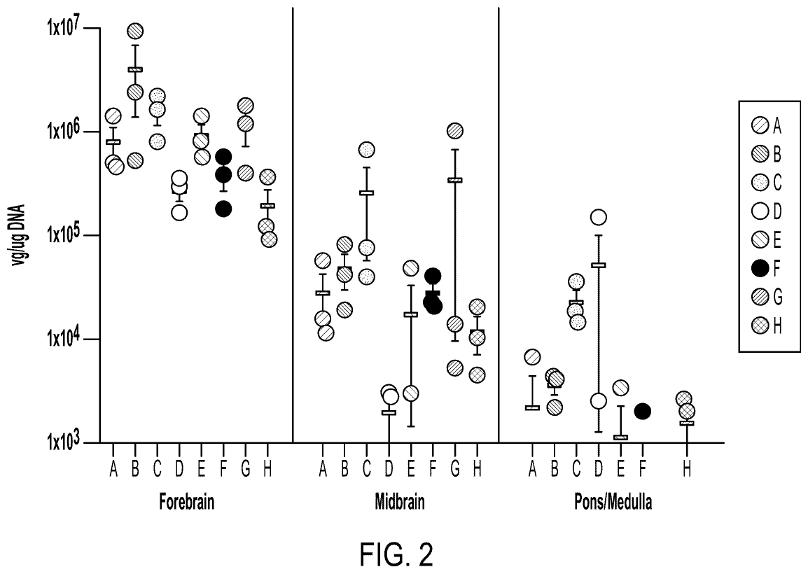

Mice were harvested at 12 weeks of age. The presence of AAV in each mouse was measured by GFP staining in sagittal sections and vector genome quantification by digital PCR (dPCR). dPCR was done by targeting the bovine growth hormone (BGH) poly A tail. Results are shown in Table 2 and FIG. 2.

TABLE 2
Vector genome (vg) quantification in the forebrain, midbrain,
and pons/medulla of mice injected with AAV variants.

| | | | | | | Ranking |
|---|---|---|---|---|---|---|
| | | Animal | | | Pons/ | (vg's |
| | Group | ID | Forebrain | Midbrain | Medulla | and GFP) |
| AAV | C | B135.7 | 1.65E+06 | 6.68E+05 | | 1 |
| Vari- | | B135.8 | 2.25E+06 | 7.69E+04 | | |
| ants | | B135.9 | 7.97E+05 | 4.07E+04 | | |
| | B | B135.4 | 2.42E+06 | 1.97E+04 | | 2 |
| | | B135.5 | 5.30E+05 | 8.38E+04 | | |
| | | B135.6 | 9.44E+06 | 4.31E+04 | | |
| | A | B135.1 | 1.43E+06 | 5.77E+04 | | 3 |
| | | B135.2 | 4.71E+05 | 1.19E+04 | | |
| | | B135.3 | 4.97E+05 | 1.58E+04 | | |
| | G | B137.4 | 4.03E+05 | 1.03E+06 | | 4 |
| | | B137.5 | 1.82E+06 | 1.42E+04 | | |
| | | B137.6 | 1.20E+06 | 5.35E+03 | | |
| | F | B137.1 | 5.79E+05 | 2.30E+04 | | 5 |
| | | B137.2 | 1.82E+05 | 4.16E+04 | | |
| | | B137.3 | 3.92E+05 | 2.12E+04 | | |
| | E | B135.13 | 8.15E+05 | 3.00E+03 | | 6 |
| | | B135.14 | 5.78E+05 | NA | | |
| | | B135.15 | 1.44E+06 | 4.96E+04 | | |
| | D | B135.10 | 2.96E+05 | 3.12E+03 | | 7 |
| | | B135.11 | 3.57E+05 | NA | | |
| | | B135.12 | 1.66E+05 | 2.84E+03 | | |
| | H | B137.7 | 9.24E+04 | 1.04E+04 | | 8 |
| | | B137.8 | 3.66E+05 | 2.09E+04 | | |
| | | B137.9 | 1.25E+05 | 4.60E+03 | | |
| | I | B137.10 | NA | NA | | |
| | | B137.11 | NA | NA | | |
| | | B137.12 | NA | NA | | |

### REFERENCES

1. Anguela, X M, and High, K A (2019). Entering the Modern Era of Gene Therapy. Annu Rev Med 70: 273-288.
2. Herzog, R W (2020). Encouraging and Unsettling Findings in Long-Term Follow-up of AAV Gene Transfer. Mol Ther 28: 341-342.
3. Perrin, G Q, Herzog, R W, and Markusic, D M (2019). Update on clinical gene therapy for hemophilia. Blood 133: 407-414.
4. Mendell, J R, Al-Zaidy, S, Shell, R, Arnold, W D, Rodino-Klapac, L R, Prior, T W, Lowes, L, Alfano, L, Berry, K, Church, K, et al. (2017). Single-Dose Gene-Replacement Therapy for Spinal Muscular Atrophy. N Engl J Med 377: 1713-1722.
5. Bennett, J, Wellman, J, Marshall, K A, McCague, S, Ashtari, M, DiStefano-Pappas, J, Elci, O U, Chung, D C, Sun, J, Wright, J F, et al. (2016). Safety and durability of effect of contralateral-eye administration of AAV2 gene therapy in patients with childhood-onset blindness caused by RPE65 mutations: a follow-on phase 1 trial. Lancet 388: 661-672.
6. Samulski, R J, and Muzyczka, N (2014). AAV-Mediated Gene Therapy for Research and Therapeutic Purposes. Annu Rev Virol 1: 427-451.
7. Nathwani, A C, Gray, J T, McIntosh, J, Ng, CY, Zhou, J, Spence, Y, Cochrane, M, Gray, E, Tuddenham, E G, and Davidoff, AM (2007). Safe and efficient transduction of the liver after peripheral vein infusion of self-complementary AAV vector results in stable therapeutic expression of human FIX in nonhuman primates. Blood 109: 1414-1421.
8. Martino, A T, Suzuki, M, Markusic, D M, Zolotukhin, I, Ryals, R C, Moghimi, B, Ertl, H C, Muruve, D A, Lee, B, and Herzog, R W (2011). The genome of self-complementary adeno-associated viral vectors increases Toll-like receptor 9-dependent innate immune responses in the liver. Blood 117: 6459-6468.
9. Verdera, H C, Kuranda, K, and Mingozzi, F (2020). AAV Vector Immunogenicity in Humans: A Long Journey to Successful Gene Transfer. Mol Ther 28: 723-746.
10. Boutin, S, Monteilhet, V, Veron, P, Leborgne, C, Benveniste, O, Montus, M F, and Masurier, C (2010). Prevalence of serum IgG and neutralizing factors against adeno-associated virus (AAV) types 1, 2, 5, 6, 8, and 9 in the healthy population: implications for gene therapy using AAV vectors. Hum Gene Ther 21: 704-712.
11. Blacklow, N R, Hoggan, M D, Kapikian, A Z, Austin, J B, and Rowe, W P (1968). Epidemiology of adenovirus-associated virus infection in a nursery population. Am J Epidemiol 88: 368-378.
12. Calcedo, R, Vandenberghe, L H, Gao, G, Lin, J, and Wilson, J M (2009). Worldwide epidemiology of neutralizing antibodies to adeno-associated viruses. J Infect Dis 199: 381-390.
13. Aronson, S J, Veron, P, Collaud, F, Hubert, A, Delahais, V, Honnet, G, de Knegt, R J, Junge, N, Baumann, U, Di Giorgio, A, et al. (2019). Prevalence and Relevance of Pre-Existing Anti-Adeno-Associated Virus Immunity in the Context of Gene Therapy for Crigler-Najjar Syndrome. Hum Gene Ther 30: 1297-1305.
14. Manno, C S, Pierce, G F, Arruda, V R, Glader, B, Ragni, M, Rasko, J J, Ozelo, M C, Hoots, K, Blatt, P, Konkle, B, et al. (2006). Successful transduction of liver in hemophilia by AAV-Factor IX and limitations imposed by the host immune response. Nat Med 12: 342-347.
15. Jiang, H, Couto, L B, Patarroyo-White, S, Liu, T, Nagy, D, Vargas, J A, Zhou, S, Scallan, C D, Sommer, J, Vijay, S, et al. (2006). Effects of transient immunosuppression on adenoassociated, virus-mediated, liver-directed gene transfer in rhesus macaques and implications for human gene therapy. Blood 108: 3321-3328.
16. Liu, Q, Huang, W, Zhang, H, Wang, Y, Zhao, J, Song, A, Xie, H, Zhao, C, Gao, D, and Wang, Y (2014). Neutralizing antibodies against AAV2, AAV5 and AAV8 in healthy and HIV-1-infected subjects in China: implications for gene therapy using AAV vectors. Gene Ther 21: 732-738.
17. Rutledge, E A, Halbert, C L, and Russell, D W (1998). Infectious clones and vectors derived from adeno-associated virus (AAV) serotypes other than AAV type 2. J Virol 72: 309-319.
18. Srivastava, A (2016). In vivo tissue-tropism of adeno-associated viral vectors. Curr Opin Virol 21: 75-80.
19. Ertl, H C J, and High, K A (2017). Impact of AAV Capsid-Specific T-Cell Responses on Design and Outcome of Clinical Gene Transfer Trials with Recombinant Adeno-Associated Viral Vectors: An Evolving Controversy. Hum Gene Ther 28: 328-337.
20. Agbandje-McKenna, M, and Kleinschmidt, J (2011). AAV capsid structure and cell interactions. Methods Mol Biol 807: 47-92.
21. Marsic, D, Govindasamy, L, Currlin, S, Markusic, D M, Tseng, Y S, Herzog, R W, Agbandje-McKenna, M, and Zolotukhin, S (2014). Vector design Tour de Force: integrating combinatorial and rational approaches to derive novel adeno-associated virus variants. Mol Ther 22: 1900-1909.
22. Li, C, and Samulski, RJ (2020). Engineering adeno-associated virus vectors for gene therapy. Nat Rev Genet 21: 255-272.
23. Lisowski, L, Dane, AP, Chu, K, Zhang, Y, Cunningham, S C, Wilson, E M, Nygaard, S, Grompe, M, Alexander, I E, and Kay, M A (2014). Selection and evaluation of clinically relevant AAV variants in a xenograft liver model. Nature 506: 382-386.
24. Paulk, N K, Pekrun, K, Zhu, E, Nygaard, S, Li, B, Xu, J, Chu, K, Leborgne, C, Dane, AP, Haft, A, et al. (2018). Bioengineered AAV Capsids with Combined High Human Liver Transduction In vivo and Unique Humoral Seroreactivity. Mol Ther 26: 289-303.
25. Zhong, L, Li, B, Mah, CS, Govindasamy, L, Agbandje-McKenna, M, Cooper, M, Herzog, R W, Zolotukhin, I, Warrington, K H, Jr., Weigel-Van Aken, K A, et al. (2008). Next generation of adeno-associated virus 2 vectors: point mutations in tyrosines lead to high-efficiency transduction at lower doses. Proc Natl Acad Sci USA 105: 7827-7832.
26. Markusic, D M, Nichols, T C, Merricks, E P, Palaschak, B, Zolotukhin, I, Marsic, D, Zolotukhin, S, Srivastava, A, and Herzog, RW (2017). Evaluation of engineered AAV capsids for hepatic factor IX gene transfer in murine and canine models. J Transl Med 15: 94.
27. Rose, J A, Maizel, J V, Jr., Inman, J K, and Shatkin, A J (1971). Structural proteins of adenovirus-associated viruses. J Virol 8: 766-770.
28. Govindasamy, L, DiMattia, M A, Gurda, B L, Halder, S, McKenna, R, Chiorini, J A, Muzyczka, N, Zolotukhin, S, and Agbandje-McKenna, M (2013). Structural insights into adeno-associated virus serotype 5. J Virol 87: 11187-11199.
29. DiMattia, M A, Nam, H J, Van Vliet, K, Mitchell, M, Bennett, A, Gurda, B L, McKenna, R, Olson, N H, Sinkovits, R S, Potter, M, et al. (2012). Structural insight into the unique properties of adeno-associated virus serotype 9. J Virol 86: 6947-6958.
30. Kapalczynska, M, Kolenda, T, Przybyla, W, Zajaczkowska, M, Teresiak, A, Filas, V, Ibbs, M, Blizniak, R, Luczewski, L, and Lamperska, K (2018). 2D and 3D cell cultures—a comparison of different types of cancer cell cultures. Arch Med Sci 14: 910-919.
31. Pekrun, K, De Alencastro, G, Luo, Q J, Liu, J, Kim, Y, Nygaard, S, Galivo, F, Zhang, F, Song, R, Tiffany, M R, et al. (2019). Using a barcoded AAV capsid library to select for clinically relevant gene therapy vectors. JCI Insight 4.
32. Grimm, D, and Zolotukhin, S (2015). E Pluribus Unum: 50 Years of Research, Millions of Viruses, and One Goal—Tailored Acceleration of AAV Evolution. Mol Ther 23: 1819-1831.
33. Vercauteren, K, Hoffman, B E, Zolotukhin, I, Keeler, G D, Xiao, J W, Basner-Tschakarjan, E, High, K A, Ertl, HC, Rice, CM, Srivastava, A, et al. (2016). Superior In vivo Transduction of Human Hepatocytes Using Engineered AAV3 Capsid. Mol Ther 24: 1042-1049.
34. Meliani, A, Leborgne, C, Triffault, S, Jeanson-Leh, L, Veron, P, and Mingozzi, F (2015). Determination of anti-adeno-associated virus vector neutralizing antibody titer with an in vitro reporter system. Hum Gene Ther Methods 26: 45-53.
35. Mingozzi, F, Chen, Y, Edmonson, S C, Zhou, S, Thurlings, R M, Tak, P P, High, K A, and Vervoordeldonk, MJ (2013). Prevalence and pharmacological modulation of humoral immunity to AAV vectors in gene transfer to synovial tissue. Gene Ther 20: 417-424.
36. Grompe, M, and Strom, S (2013). Mice with human livers. Gastroenterology 145: 1209-1214.
37. Bissig-Choisat, B, Wang, L, Legras, X, Saha, P K, Chen, L, Bell, P, Pankowicz, F P, Hill, M C, Barzi, M, Leyton, C K, et al. (2015). Development and rescue of human familial hypercholesterolaemia in a xenograft mouse model. Nat Commun 6: 7339.
38. Michailidis, E, Vercauteren, K, Mancio-Silva, L, Andrus, L, Jahan, C, Ricardo-Lax, I, Zou, C, Kabbani, M, Park, P, Quirk, C, et al. (2020). Expansion, in vivo-ex vivo cycling, and genetic manipulation of primary human hepatocytes. Proc Natl Acad Sci USA 117: 1678-1688.
39. Zou, C, Vercauteren, K O A, Michailidis, E, Kabbani, M, Zoluthkin, I, Quirk, C, Chiriboga, L, Yazicioglu, M, Anguela, X M, Meuleman, P, et al. (2020). Experimental Variables that Affect Human Hepatocyte AAV Transduction in Liver Chimeric Mice. Mol Ther Methods Clin Dev 18: 189-198.
40. Vanwolleghem, T, Libbrecht, L, Hansen, B E, Desombere, I, Roskams, T, Meuleman, P, and Leroux-Roels, G (2010). Factors determining successful engraftment of hepatocytes and susceptibility to hepatitis B and C virus infection in uPA-SCID mice. J Hepatol 53: 468-476.
41. Kawahara, T, Toso, C, Douglas, D N, Nourbakhsh, M, Lewis, J T, Tyrrell, D L, Lund, G A, Churchill, T A, and Kneteman, N M (2010). Factors affecting hepatocyte isolation, engraftment, and replication in an in vivo model. Liver Transpl 16: 974-982.
42. Billerbeck, E, Mommersteeg, M C, Shlomai, A, Xiao, J W, Andrus, L, Bhatta, A, Vercauteren, K, Michailidis, E, Dorner, M, Krishnan, A, et al. (2016). Humanized mice efficiently engrafted with fetal hepatoblasts and syngeneic immune cells develop human monocytes and NK cells. J Hepatol 65: 334-343.
43. Ling, C, Wang, Y, Zhang, Y, Ejjigani, A, Yin, Z, Lu, Y, Wang, L, Wang, M, Li, J, Hu, Z, et al. (2014). Selective in vivo targeting of human liver tumors by optimized AAV3 vectors in a murine xenograft model. Hum Gene Ther 25: 1023-1034.
44. Li, S, Ling, C, Zhong, L, Li, M, Su, Q, He, R, Tang, Q, Greiner, D L, Shultz, L D, Brehm, M A, et al. (2015). Efficient and Targeted Transduction of Nonhuman Primate Liver With Systemically Delivered Optimized AAV3B Vectors. Mol Ther 23: 1867-1876.
45. Baruteau, J, Waddington, S N, Alexander, I E, and Gissen, P (2017). Gene therapy for monogenic liver diseases: clinical successes, current challenges and future prospects. J Inherit Metab Dis 40: 497-517.
46. de Alencastro, G, Pekrun, K, Valdmanis, P, Tiffany, M, Xu, J, and Kay, M A (2020). Tracking Adeno-Associated Virus Capsid Evolution by High-Throughput Sequencing. Hum Gene Ther 31: 553-564.
47. Ran, G, Chen, X, Xie, Y, Zheng, Q, Xie, J, Yu, C, Pittman, N, Qi, S, Yu, F X, Agbandje-McKenna, M, et al. (2020). Site-Directed Mutagenesis Improves the Transduction Efficiency of Capsid Library-Derived Recombinant AAV Vectors. Mol Ther Methods Clin Dev 17: 545-555.
48. Zhong, L, Li, B, Jayandharan, G, Mah, C S, Govindasamy, L, Agbandje-McKenna, M, Herzog, R W, Weigel-Van Aken, K A, Hobbs, J A, Zolotukhin, S, et al. (2008). Tyrosine-phosphorylation of AAV2 vectors and its consequences on viral intracellular trafficking and transgene expression. Virology 381: 194-202.
49. Markusic, D M, Herzog, R W, Aslanidi, G V, Hoffman, B E, Li, B, Li, M, Jayandharan, G R, Ling, C, Zolotukhin, I, Ma, W, et al. (2010). High-efficiency transduction and correction of murine hemophilia B using AAV2 vectors devoid of multiple surface-exposed tyrosines. Mol Ther 18: 2048-2056.
50. Martino, A T, Basner-Tschakarjan, E, Markusic, D M, Finn, J D, Hinderer, C, Zhou, S, Ostrov, D A, Srivastava, A, Ertl, H C, Terhorst, C, et al. (2013). Engineered AAV vector minimizes in vivo targeting of transduced hepatocytes by capsid-specific CD8+ T cells. Blood 121: 2224-2233.
51. Buning, H, and Srivastava, A (2019). Capsid Modifications for Targeting and Improving the Efficacy of AAV Vectors. Mol Ther Methods Clin Dev 12: 248-265.
52. Shirley, J L, de Jong, Y P, Terhorst, C, and Herzog, RW (2020). Immune Responses to Viral Gene Therapy Vectors. Mol Ther 28: 709-722.
53. Shirley, J L, Keeler, G D, Sherman, A, Zolotukhin, I, Markusic, D M, Hoffman, B E, Morel, L M, Wallet, M A, Terhorst, C, and Herzog, R W (2020). Type I IFN Sensing by cDCs and CD4(+) T Cell Help Are Both Requisite for Cross-Priming of AAV Capsid-Specific CD8(+) T Cells. Mol Ther 28: 758-770.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be illustrative examples, and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A variant recombinant adeno-associated virus (rAAV) serotype 5 (AAV5) capsid protein comprising mutations in each of amino acid residues 377 and 378 in the wild-type AAV5 VP1 sequence of SEQ ID NO: 1.

2. The capsid protein of claim 1, wherein the protein comprises an N378K substitution.

3. The capsid protein of claim 1 or 2, wherein the protein comprises an E377D substitution.

4. The capsid protein of claim 1 or 2, wherein the protein comprises an E377G substitution.

5. The capsid protein of any one of claims 1-3, wherein the protein comprises the amino acid sequence of SEQ ID NO: 2 (AAV5-DK).

6. The capsid protein of any one of claims 1, 2 and 4, wherein the protein comprises the amino acid sequence of SEQ ID NO: 3 (AAV5-GK).

7. A variant recombinant adeno-associated virus (rAAV) serotype 5 (AAV5) capsid protein comprising either of the following sequences:
(a) LFRFVSTDATGNLKF (SEQ ID NO: 6) in variable region (VR) IV, or
(b) LSAGGNRNYLSAKA (SEQ ID NO: 7) in VR V, of AAV5 VP1 protein.

8. A variant recombinant adeno-associated virus (rAAV) serotype 5 (AAV5) capsid protein comprising mutations in at least two, at least three, at least four, at least five, or at least six of amino acid residues 436, 442, 443, 446, 447, and 448 in the wild-type AAV5 VP1 sequence of SEQ ID NO: 1.

9. The capsid protein of claim 8, wherein the protein comprises mutations at each of amino acid residues 436, 442, 443, 446, 447, and 448.

10. The capsid protein of claim 8, wherein the protein comprises at least two, at least three, at least four, at least five, or at least six of the following substitutions: Y436F, N442D, N443A, G446N, V447L, and Q448K.

11. The capsid protein of any one of claims 8-10, wherein the protein comprises each of the following substitutions: Y436F, N442D, N443A, G446N, V447L, and Q448K.

12. The capsid protein of any one of claims 8-11, wherein the protein comprises the amino acid sequence of SEQ ID NO: 4 (AAV5-FDA).

13. A variant recombinant adeno-associated virus (rAAV) serotype 5 (AAV5) capsid protein comprising mutations in at least two, at least three, at least four, at least five, at least six, or at least seven of amino acid residues 478, 479, 481, 484, 485, 486 and 489 in the wild-type AAV5 VP1 sequence of SEQ ID NO: 1.

14. The capsid protein of claim 13, wherein the protein comprises mutations at each of amino acid residues 478, 479, 481, 484, 485, 486 and 489.

15. The capsid protein of claim 13, wherein the protein comprises at least two, at least three, at least four, at least five, or at least six of the following substitutions: G478S, S479A, V481G, A484N, S485Y, V486L, and F489K.

16. The capsid protein of any one of claims 13-15, wherein the protein comprises each of the following substitutions: G478S, S479A, V481G, A484N, S485Y, V486L, and F489K.

17. The capsid protein of any one of claims 13-16, wherein the protein comprises the amino acid sequence of SEQ ID NO: 5 (AAV5-SAG).

18. A variant recombinant AAV5 particle comprising the recombinant AAV capsid protein of any one of claims 1-17.

19. The variant recombinant AAV5 particle of claim 18, further comprising a nucleic acid comprising a transgene of interest.

20. The variant recombinant AAV5 particle of claim 18 or 19, wherein the nucleic acid is single stranded.

21. The variant recombinant AAV5 particle of claim 18 or 19, wherein the nucleic acid is self-complementary.

22. A composition comprising a plurality of the variant recombinant AAV5 particle of any one of claims 18-21.

23. The composition of claim 22 further comprising a pharmaceutically acceptable carrier.

24. The composition of claim 22 or 23, wherein the plurality is in an amount of between 1×10¹¹ vgs/ml and 2×10¹¹ vgs/ml, or between 1×10¹² and 4×10¹² vgs/ml.

25. A cell comprising a plurality of the variant recombinant AAV5 particle of any one of claims 18-21.

26. The cell of claim 25, wherein the cell is a mesenchymal stem cell.

27. A method of transducing a mesenchymal stem cell with a transgene of interest, the method comprising providing to the cell the variant recombinant AAV particle of any one of claims 18-21 or the composition of any one of claims 22-24.

28. The method of claim 27, wherein the method provides about a 15%, a 30%, a 50%, a 100%, a 200%, a 300%, a 400%, a 500%, a 750%, or a 1000% increase in transduction of the transgene of interest in the mesenchymal stem cell, relative to a wild-type recombinant AAV5 particle.

29. The method of claim 27 or 28, wherein the mesenchymal stem cell is derived from a mammalian subject.

30. A method of treating a disease or disorder comprising administering the variant recombinant AAV particle of any one of claims 18-21, the composition of any one of claims 22-24, or the cell of claim 25 or 26, to a subject in need thereof.

31. The method of claim 30, wherein the subject is a mammal.

32. The method of any one of claims 29-31, wherein the subject is human.

33. The method of any one of claims 30-32 further comprising re-administering the recombinant AAV particle, the composition, or the cell to the subject.

34. A method of administering the variant recombinant AAV particle of any one of claims 18-21, the composition of any one of claims 22-24, or the cell of claim 25 or 26, to a subject in need thereof who has previously been administered the recombinant AAV particle, the composition, or the cell.

35. A method of transducing a neuron or glial cell with a transgene of interest, the method comprising providing to the cell the variant recombinant AAV particle of any one of claims 18-21 or the composition of any one of claims 22-24.

36. The method of claim 27, wherein the method provides about a 15%, a 30%, a 50%, a 100%, a 200%, a 300%, a 400%, a 500%, a 750%, or a 1000% increase in transduction of the transgene of interest in the neuron or glial cell, relative to a wild-type recombinant AAV5 particle.

37. The variant recombinant AAV particle of any one of claims 18-21, the composition of any one of claims 22-24, or the cell of claim 25 or 26, for use as a medicament.

38. A polynucleotide encoding the capsid of any one of claims 1-17.

39. A host cell comprising the polynucleotide of claim 38.
